SEQUENCE LISTING (3 sequences)

SEQ ID NO: 1 - DNA, 20 bp, Artificial Sequence
  Chemically Synthesized
tagcggataacacagtcacc

SEQ ID NO: 2 - DNA, 20 bp, Artificial Sequence
  Chemically Synthesized
tagcggataacacagtcacc

SEQ ID NO: 3 - DNA, 20 bp, Artificial Sequence
  Chemically Synthesized
tagcggataacacagtcacc

(19) United States
(12) Patent Application Publication
(10) Pub. No.: US 2006/0228703 A1
(43) Pub. Date: Oct. 12, 2006

(54) Displacement assay for the detection of nucleic acid oligomer hybridization events

(76) Inventors: Gerhard Hartwich, Munchen (DE); Peter Frischmann, Munchen (DE); Ute Haker, Grobenzell (DE); Herbert Wieder, Munchen (DE)

Correspondence Address: CROCKETT & CROCKETT, 24012 CALLE DE LA PLATA, SUITE 400, LAGUNA HILLS, CA, 92653, US

(21) Appl. No.: 10/487,816

(22) PCT Filed: Aug. 26, 2002
(86) PCT No.: PCT/DE02/03122

§ 371 Date: Sep. 8, 2005

(30) Foreign Application Priority Data
Aug. 25, 2001 (DE) 101 41 691.1
Apr. 6, 2002 (WO) PCT/DE02/01269

(51) Int. Cl. C12Q 1/68

(52) U.S. Cl. 435/6

(57) ABSTRACT

Described is a method for detection of nucleic acid oligomer hybridization events, the method comprising the steps: providing a modified surface, the modification consisting in the attachment of at least one type of ligate nucleic acid oligomer; providing signal nucleic acid oligomer ligands; providing a sample having nucleic acid oligomer ligands; bringing a defined quantity of the signal nucleic acid oligomer ligands into contact with the modified surface and bringing the sample and the nucleic acid oligmer ligands contained therein into contact with the modified surface; detecting the signal nucleic acid oligomer ligands; and comparing with reference values the values obtained from the detection of the signal nucleic acid oligomer ligands.

### FIELD OF THE INVENTION

The present invention is directed to a method for detection of nucleic acid oligomer hybridization events.

### BACKGROUND OF THE INVENTION

Immunoassays and, increasingly, also DNA and RNA sequence analysis are being employed in disease diagnosis, toxicological test procedures, genetic research and development, and in the agricultural and pharmaceutical sectors. In addition to the known serial methods using autoradiographical or optical detection, increasingly, parallel detection methods by means of array technology using what are known as DNA or protein chips are being applied. For the parallel methods, too, the actual detection is based on either optical, radiographical, mass spectrometric or electrochemical methods.

In addition to their applications for sequencing, oligonucleotide or DNA chips can also be used for SNP (single nucleotide polymorphism) or gene expression analysis, since they allow the activity level of a large number of individual active genes (cDNA or mRNA) of a specific cell type or tissue to be measured in parallel which is possible with conventional (serial) gene detection methods only with difficulty or at great expense. The analysis of pathologically modified gene activities, in turn, can contribute to clarification of disease mechanisms and identification of new points of attack for therapeutic application. In addition, (only) DNA chips allow so-called pharmacogenomic studies during clinical development, which can significantly increase the effectiveness and safety of the drugs. The pharmacogenomic studies focus on the question of which genetic factors are responsible for patients displaying differing reactions to the same drug. Extensive polymorphism analyses (base-pair mismatch analyses) of genes that encode important metabolic enzymes can uncover answers to such questions.

To analyze genes on a chip, a library of known DNA sequences (“probe oligonucleotides”) is attached to a surface in an ordered grid such that the position of each individual DNA sequence is known. If fragments of active genes (“target oligonucleotides”) whose sequences are complementary to specific probe oligonucleotides on the chip exist in the test solution, the target oligonucleotides can be identified (read) by detecting the appropriate hybridization events on the chip.

Protein chips whose test sites carry specific antigen (or antibody) probes instead of probe oligonucleotides can be employed in proteome analysis or in parallelization of diagnostics.

The use of radioactive labels in DNA/RNA sequencing is associated with several disadvantages, such as elaborate, legally required safety precautions in dealing with radioactive materials. For fluorescence and mass spectrometric detection, the cost of equipment is very high. Some of the disadvantages of labeling with radioactive elements or fluorescent dyes can be avoided if association events are detected based on the associated change in the electrochemical properties (cf. WO 97/46568, WO 99/51778, WO 00/31101, WO 00/42217).

As regards DNA analysis, it is thus desirable and, for the user, advantageous when the targets (DNA fragment) need not be modified with a detection label.

Thus, although there are options for detecting nucleic acid oligomer hybrids, there is great need for simple, economical, and reliable detection principles that can be carried out easily, especially in the area of lower-density arrays (low-density DNA chips with few to a few hundred test sites per cm², e.g. for so-called POC (point-of-care) systems).

### DESCRIPTION OF THE INVENTION

Therefore, it is the object of the present invention to create for detection of nucleic acid oligomer hybridization events a method that does not exhibit the disadvantages of the background art.

This object is solved by the method according to independent claim 1 and the kit according to independent claim 48. Further advantageous details, aspects and designs of the present invention are evident from the dependent claims, the description, the drawings and the examples.

The following abbreviations and terms will be used in the context of the present invention:
DNA deoxyribonucleic acid
RNA ribonucleic acid
PNA peptide nucleic acid (synthetic DNA or RNA in which the sugar-phosphate moiety is replaced by an amino acid. If the sugar-phosphate moiety is replaced by the —NH—(CH₂)₂—N(COCH₂-base)-OH₂CO— moiety, PNA will hybridize with DNA.)
A adenine
G guanine
C cytosine
T thymine
U uracil
base A, G, T, C or U
bp base pair
nucleic acid At least two covalently-joined nucleotides or at least two covalently-joined pyrimidine (e.g. cytosine, thymine or uracil) or purine bases (e.g. adenine or guanine). The term nucleic acid refers to any backbone of the covalently-joined pyrimidine or purine bases, such as the sugar-phosphate backbone of DNA, cDNA or RNA, a peptide backbone of PNA, or analogous structures (e.g. a phosphoramide, thiophosphate or dithiophosphate backbone). An essential feature of a nucleic acid within the meaning of the present invention is that it can sequence-specifically bind naturally occurring cDNA or RNA.
nt nucleotide
nucleotide monomer building block of a nucleic acid oligomer
nucleic acid oligomer Nucleic acid of a base length that is not further specified (e.g. nucleic acid octamer: a nucleic acid having any backbone in which eight pyrimidine or purine bases are covalently bound to each other).
na oligomer nucleic acid oligomer
oligomer Equivalent to nucleic acid oligomer.
oligonucleotide Equivalent to oligomer or nucleic acid oligomer, e.g. a DNA, PNA or RNA fragment of a base length that is not further specified.
oligo Abbreviation for oligonucleotide.
sequence A succession of nucleotides in a nucleic acid oligomer.
complementary To form the Watson-Crick double-stranded nucleic acid oligomer structure, the two single-strands associate, i.e. hybridize, the nucleotide sequence of one strand being complementary to the nucleotide sequence of the other strand, such that the base A (or C) of one strand forms hydrogen bonds with the base T (or G) of the other strand (in RNA, T is replaced by uracil).
mismatch To form the Watson-Crick double-stranded nucleic acid oligomer structure, the two single strands hybridize in such a way that the base A (or C) of one strand forms hydrogen bonds with the base T (or G) of the other strand (in RNA, T is replaced by uracil). Any other base pairing within the hybrid does not form hydrogen bonds, distorts the structure and is referred to as a “mismatch”.
perfect match A hybrid of two complementary nucleic acid oligomers in which no mismatch appears.
ss single strand
ds double strand
oxidizing agent A chemical compound (chemical substance) that oxidizes another chemical compound (chemical substance) by taking up electrons from this other chemical compound (chemical substance).
reducing agent A chemical compound (chemical substance) that reduces another chemical compound (chemical substance) by giving up electrons to this other chemical compound (chemical substance).
redox-active Refers to the property of a moiety of giving up electrons to a suitable oxidizing agent or taking up electrons from a suitable reducing agent under certain external conditions.
EDTA ethylenediamine tetraacetate (sodium salt)
sulfo-NHS N-hydroxysulfosuccinimide
NHS N-hydroxysuccinimide
EDC (3-dimethylaminopropyl)-carbodiimide
HEPES N-[2-hydroxyethyl]piperazine-N′-ethanesulfonic acid]
Tris trishydroxymethylamino methane
ligand Refers to nucleic acid oligomer ligands that are specifically bound by ligate nucleic acid oligomers.
ligate Refers to ligate nucleic acid oligomers.
linker A molecular link between two molecules or between a surface atom, surface molecule or surface molecule group and another molecule. Linkers can usually be purchased in the form of alkyl, alkenyl, alkynyl, heteroalkyl, heteroalkenyl or heteroalkynyl chains, the chain being derivatized in two places with (identical or different) reactive groups. These groups form a covalent chemical bond in simple/known chemical reactions with the appropriate reaction partner. The reactive groups may also be photoactivatable, i.e. the reactive groups are activated only by light of a specific or any given wavelength. Preferred linkers are those having a chain length of 1-20, especially a chain length of 1-14, the chain length here representing the shortest continuous link between the structures to be joined, in other words between the two molecules or between a surface atom, surface molecule or surface molecule group and another molecule.
spacer Equivalent to linker.
mica Muscovite lamina, a support material for the application of thin films.

Au—S—(CH₂)₂-ss-digo Gold film on mica having a covalently applied monolayer comprising derivatized single-strand oligonucleotide. Here, the oligonucleotide's terminal phosphate group at the 3′-end is esterified with (HO—(CH₂)₂-S)₂ to form P—O—(CH₂)₂—S—S—(CH₂)₂—OH, the S—S bond being homolytically cleaved and producing one Au—S—R bond each.
Au—S—(CH₂)₂-ds-digo A—S—(CH₂)₂-ss-digo-spacer hybridized with the oligonucleotide that is complementary to the ss-oligo.
K_{A} The association constant for the association of ligate and ligand or ligate and signal ligand.
E The electrode potential on the working electrode
E_{Ox} The potential at maximum current of the oxidation of a reversible electrooxidation or electroreduction.
i current density (current per cm² electrode surface)
cyclic voltammetry Recording a current-voltage curve. Here, the potential of a stationary working electrode is changed linearly as a function of time, starting at a potential at which no electrooxidation or electroreduction occurs, up to a potential at which a species that is dissolved or adsorbed to the electrode is oxidized or reduced (i.e. a current flows). Following completion of the oxidation or reduction operation, which produces in the current-voltage curve an initially increasing current and, after reaching maximum, a gradually decreasing current, the direction of the potential scan is reversed. The behavior of the products of electrooxidation or electroreduction is then recorded in a reverse run.
amperometry Recording a current-time curve. Here, the potential of a working electrode is set, for example by a potential jump, to a potential at which the electrooxidation or electroreduction of a dissolved or adsorbed species occurs, and the flowing current is recorded as a function of time.
chronocoulometry Recording a charge-time curve. Here, the potential of a working electrode is set, for example by a potential jump, to a potential at which the electrooxidation or electroreduction of a dissolved or adsorbed species occurs, and the transferred charge is recorded as a function of time. Chronocoulometry can thus be understood as an integral of amperometry.
SECM scanning electrochemical microscopy

The present invention provides a method for detection of nucleic acid oligomer hybridization events, comprising the steps: providing a modified surface, the modification consisting in the attachment of at least one type of ligate nucleic acid oligomer; providing signal nucleic acid oligomer ligands; providing a sample having nucleic acid oligomer ligands; bringing a defined quantity of the signal nucleic acid oligomer ligands into contact with the modified surface and bringing the sample and the nucleic acid oligmer ligands contained therein into contact with the modified surface; detecting the signal nucleic acid oligomer ligands; and comparing with reference values the values obtained from the detection of the signal nucleic acid oligomer ligands.

Bringing a defined quantity of the signal nucleic acid oligomer ligands into contact with the modified surface and bringing the sample into contact with the modified surface may, in principle, also take place simultaneously, but preferably the signal nucleic acid oligomer ligands and the sample are brought into contact separately.

If a defined quantity of the signal nucleic acid oligomer ligands is brought into contact with the modified surface and, separately, the sample is brought into contact with the modified surface, there are two alternatives, namely bringing a defined quantity of the signal nucleic acid oligomer ligands into contact with the modified surface and thereafter bringing the sample into contact with the modified surface, on the one hand, and on the other hand, first bringing the sample into contact with the modified surface and thereafter bringing a defined quantity of the signal nucleic acid oligomer ligands into contact with the modified surface. The present invention comprises both alternatives.

If a defined quantity of the signal nucleic acid oligomer ligands is brought into contact with the modified surface before the sample is brought into contact with the modified surface, according to a preferred embodiment of the present invention, the reference values may be redetermined each time before the sample is brought into contact with the modified surface. For this purpose, after a defined quantity of the signal nucleic acid oligomer ligands is brought into contact with the modified surface, a detection of the signal nucleic acid oligomer ligands is carried out and only thereafter is the sample brought into contact with the modified surface. Thereafter, the signal nucleic acid oligomer ligands are detected a second time, and the values determined in the second detection are compared with the reference values determined in the first detection.

According to a particularly preferred embodiment, after the first detection of the signal nucleic acid oligomer ligands, the modified surface is washed and, after the sample is brought into contact with the modified surface, the same defined quantity of signal nucleic acid oligomer ligands as before the first detection (reference measurement) is once more brought into contact with the modified surface. Only thereafter is the second detection of the signal nucleic acid oligomer ligands carried out.

According to a further, particularly preferred embodiment, after the first detection of the signal nucleic acid oligomer ligands, before and/or during the washing of the modified surface, conditions are set or actions taken that lead to at least predominant dissociation of ligate nucleic acid oligomers and signal nucleic acid oligomer ligands. In this way, during the wash step, as large a portion of the signal nucleic acid oligomer ligands as possible is removed from the surface.

In order to achieve the at least predominant dissociation of ligate nucleic acid oligomers and signal nucleic acid oligomer ligands, the temperature may be raised above the melting temperature of the double-stranded oligonucleotides consisting of ligate nucleic acid oligomer and signal nucleic acid oligomer ligand, chaotropic salts may be added or a potential that lies above the electrostringent potential may be applied.

Alternatively, according to a preferred embodiment of the present invention, the reference values may be determined after the modified surface has been brought into contact, simultaneously or separately, with nucleic acid oligomer ligands and signal nucleic acid oligomer ligands and the subsequent detection of the signal nucleic acid oligomer ligands. For this reference value determination, first, conditions are set or actions taken that lead to at least predominant dissociation of ligate nucleic acid oligomers and nucleic acid oligomer ligands and to at least predominant dissociation of ligate nucleic acid oligomers and signal nucleic acid oligomer ligands, then the modified surface is washed, thereafter signal nucleic acid oligomer ligands are brought into contact with the modified surface, the same defined quantity of signal nucleic acid oligomer ligands being used as for the first addition, and finally, the signal nucleic acid oligomer ligands are detected and the reference values thus determined. This method of reference value determination is particularly preferred when the sample is brought into contact with the modified surface before a defined quantity of the signal nucleic acid oligomer ligands is brought into contact with the modified surface.

One of these conditions that also lead to increased dissociation of ligate nucleic acid oligomers and nucleic acid oligomer ligands, mentioned in connection with the dissociation of ligate nucleic acid oligomers and signal nucleic acid oligomer ligands, is the addition of chaotropic salts, advantageously in a concentration of at least 3 mol/l. A temperature increase above the melting temperature of the double-stranded oligonucleotides consisting of ligate nucleic acid oligomers and nucleic acid oligomer ligands, and above the melting temperature of the double-stranded oligonucleotides consisting of ligate nucleic acid oligomers and signal nucleic acid oligomer ligands, also leads to increased dissociation of the double-strand oligomers. Preferably, the temperature is increased to at least 5° C. above the melting temperature. Applying a potential that lies above the electrostringent potential may also be considered as a further action, especially an increase in the (negative) potential by at least 10 mV above the electrostringent potential, and in addition, treatment with NaOH.

The melting temperature of the double-strand comprising ligate nucleic acid oligomers and signal nucleic acid oligomer ligands, or of the double-strand comprising ligate nucleic acid oligomers and nucleic acid oligomer ligands, may either be determined under the appropriate external conditions, in other words with the given concentration of oligonucleotides, a specific salt content, a specific salt, in the presence of dissociation-promoting organic solvents such as formamide, DMF, etc., or calculated relatively precisely in broad ranges (see e.g. the oligo analyzer available on the Internet, www.idtdna.com/program/main/home.asp).

For electrode-immobilized ligate nucleic acid oligomers having an associated counter strand, the electrode potential may be used to displace the polyanionic counter strand from the electrode. For negative to slightly positive potentials (E<0.2 V versus silver wire), due to the repulsion of the negatively charged DNA backbone, both single- and double-stranded nucleic acid oligomers attached to a conductive surface are present in a rather stretched conformation. If the potential is shifted further into the negative, the surface becomes more negatively charged, and less strongly bound nucleic acid oligomer ligands and signal nucleic acid oligomer ligands, i.e. those that exhibit one or more base mismatches in the binding region, are displaced from the modified surface. As described for the melting temperature, this so-called electrostringent potential, too, may be determined experimentally for certain external parameters.

Examples of anions of chaotropic salts for dissociating double-strand oligonucleotides include CCl₃COO⁻, CNS⁻, CF₃COO⁻, ClO₄⁻, I⁻, (see also Robinson and Grant, Journal of Biological Chemistry, 241, 1966, p. 1329ff and Kessler et al., U.S. Pat. No. 5,753,433). Chaotropic salts lower the melting temperature.

In all of the methods described in the context of the present invention, to improve quantification of the measured values, a correction value that corresponds to the background signal of the modified surface used may be determined for each measured value. The correction value is generally subtracted from the detection and reference signals to improve quantification of the detection.

In the context of the present invention, the determination of a correction value for modified surfaces that are not associated with nucleic acid oligomer ligands and signal nucleic acid oligomer ligands may in principle take place at three different points in time: a) after the sample and the nucleic acid oligomer ligands contained therein are brought into contact with the modified surface but before the signal nucleic acid oligomer ligands are added; b) before the sample and the nucleic acid oligomer ligands contained therein are brought into contact with the modified surface and before the signal nucleic acid oligomer ligands are added; c) after the double-strand hybrids comprising ligate nucleic acid oligomer and nucleic acid oligomer ligand, or comprising ligate nucleic acid oligomer and signal nucleic acid oligomer ligand, have dissociated, after the modified surface is washed in the absence of signal nucleic acid oligomer ligands.

When carrying out the method according to the present invention, the determination of the correction values may also take place several times, at the same point in time or at the above-described different points in time. The measured value used each time is then determined by forming the arithmetic mean from the various measurements for the relevant region.

According to the particularly preferred embodiments of the present invention described below, the separate execution of a reference measurement can be avoided. In these cases, on the modified surface are applied reference sites to which, after the sample is added, a very specific level of association can be assigned. The signal obtained from the detection is then characteristic for this specific level of association and can be consulted to standardize the signals of the test sites.

All of the methods described in the context of the present invention may namely also be carried out using a modified surface, the surface having been modified by attaching at least two types of ligate nucleic acid oligomers. The differing types of ligate nucleic acid oligomers are bound to the surface in spatially substantially separate regions. The term “substantially separate regions” is understood to mean regions on the surface that are quite predominantly modified by attaching a specific type of ligate. Merely in areas in which two such substantially separate regions adjoin can it happen that differing types of ligate nucleic acid oligomers commingle. In the method preferred in the context of the present invention, before the sample is brought into contact with the modified surface, one type of nucleic acid oligomer ligand is added to the sample, the nucleic acid oligomer ligand being a binding partner having a high association constant to a specific type of ligate nucleic acid oligomer that is present bound to the surface in a specific region (test site T₁₀₀). This added type of nucleic acid oligomer ligand does not associate, or associates only negligibly, to the remaining types of ligate nucleic acid oligomers that are bound to the surface. Here, the nucleic acid oligomer ligand is added to the sample in a quantity that is greater than the quantity of nucleic acid oligomer ligands needed to completely associate the ligate nucleic acid oligomers of the T₁₀₀ test sites. The last step of this method—after the sample is brought into contact with the modified surface, the sample containing the type of nucleic acid oligomer ligand having a high association constant to the ligate nucleic acid oligomers bound in the T₁₀₀ region, optional stringent hybridization, and after the signal nucleic acid oligomer ligands are brought into contact with the modified surface—is comparing with the value obtained for the T₁₀₀ region the values obtained from the detection of the signal nucleic acid oligomer ligands. The value obtained for the T₁₀₀ region thus corresponds to the value for complete association (100%).

According to a particularly preferred embodiment, a modified surface is used that was modified by attaching at least three types of ligate nucleic acid oligomers. The differing types of ligate nucleic acid oligomers are bound to the surface in spatially substantially separate regions. Here, in a specific region (test site T₀) is bound to the surface at least one type of ligate nucleic acid oligomer, about which it is known that no binding partner having a high association constant is contained in the sample, that is, the complementary nucleic acid oligomer ligand does not occur in the sample. In this particularly preferred method, too, before the sample is brought into contact with the modified surface, one type of nucleic acid oligomer ligands is added to the sample, the added type of nucleic acid oligomer ligand being a binding partner having a high association constant to a specific type of ligate nucleic acid oligomer that is present bound to the surface in a specific region (test site T₁₀₀). This added type of nucleic acid oligomer ligand does not associate, or associates only negligibly, to the remaining types of ligate nucleic acid oligomers bound to the surface. Here, the nucleic acid oligomer ligand is added to the sample in a quantity that is greater than the quantity of nucleic acid oligomer ligands needed to completely associate the ligate nucleic acid oligomers of the T₁₀₀ test sites. The last step of this method—after the sample is brought into contact with the modified surface, the sample containing the type of nucleic acid oligomer ligand having a high association constant to the ligate nucleic acid oligomers bound in the T₁₀₀ region, optional stringent hybridization, and after the signal nucleic acid oligomer ligands are brought into contact with the modified surface—is comparing the values obtained from the detection of the signal nucleic acid oligomer ligands with the value obtained for the T₁₀₀ region and with the value obtained for the T₀ region. The value obtained for the T₀ region thus corresponds to the value for no association (0%).

According to a most particularly preferred embodiment of the two above-described methods, which do without a separate reference measurement, at least one additional type of nucleic acid oligomer ligand is added to the sample before the sample is brought into contact with the modified surface, it being known that this nucleic acid oligomer ligand is not contained in the original sample. This additional type of nucleic acid oligomer ligand exhibits an association constant>0 to one type of ligate nucleic acid oligomer that is present bound to the surface in a specific region (test site Tₙ). The nucleic acid oligomer ligand is added to the sample in such a quantity that, after the sample is brought into contact with the modified surface, n % of the ligate nucleic acid oligomers of the Tₙ test site are present in associated form. The last step of this method—after the sample is brought into contact with the modified surface, the sample containing the types of nucleic acid oligomer ligands that associate to the ligate nucleic acid oligomers bound in the T₁₀₀ and Tₙ regions, optional stringent hybridization, and after the signal nucleic acid oligomer ligands are brought into contact with the modified surface—is comparing the values obtained from the detection of the signal nucleic acid oligomer ligands with the value obtained for the T₁₀₀ region, with the value obtained for the T₀ region and with the values obtained for the Tₙ regions. The value obtained for a specific test site Tₙ thus corresponds to the value for the presence of n % ligate-ligand associates based on the total number of ligate nucleic acid oligomers of the appropriate type.

The quantity of nucleic acid oligomer ligand that must be brought into contact with the modified surface to effect an n % association on the Tₙ test site can be determined by those of ordinary skill in the art through simple routine analyses. For this purpose, e.g. after detecting the values for T₀ and T₁₀₀, a calibrated measurement is carried out, in which is determined the signal intensity of (different) detection labels with which the ligate nucleic acid oligomer and the nucleic acid oligomer ligand are equipped. The ratio of ligand-label signal to ligate-label signal is n %.

In a particularly preferred form of reference value determination by establishing T₀, Tₙ and T₁₀₀ values, mixtures comprising n % ligate nucleic acid oligomers that are used for the T₁₀₀ test site, and (100−n)% ligate nucleic acid oligomers that are used for the T₀ test site, are used as ligate nucleic acid oligomers for the modification of the Tₙ measurement regions. In this case, the establishment of suitable Tₙ nucleic acid oligomer ligands and their addition to the sample before the sample is brought into contact with the modified surface can be omitted if adequate T₁₀₀ nucleic acid oligomer ligand is added to the sample, such that not only the T₁₀₀ test site can completely associate with T₁₀₀ nucleic acid oligomer ligand, but also the relevant n % of the Tₙ test sites can associate with the T₁₀₀ nucleic acid oligomer ligand. If a sufficient number of Tₙ reference sites is applied on the modified surface, a reference curve can be recorded with great precision: Standardizing the measurements of the actual test sites with the aid of this reference curve significantly improves the reproducibility of chip-technology-aided analyses.

When carrying out the methods according to the present invention, the measured values for the T₀, Tₙ and T₁₀₀ regions may also be determined several times, at the same point in time or at the above-described different points in time. The measured value used each time is then determined by forming the arithmetic mean from the various measurements for the relevant region. The measured values for the T₀, Tₙ and T₁₀₀ regions can, as described above, be improved by establishing correction values or arithmetically averaged correction values. In this case, it is advantageous to improve also the measured values of all other regions of the modified surface (test region M) through correction values, mathematical methods of determination being preferred that are identical for the establishment of the corrected measured values for the T₀, Tₙ and T₁₀₀ regions and for the actual measurement region M (identical standardization).

It should be pointed out that the discovery of a nucleic acid oligomer ligand that is not contained in the sample is no problem whatsoever, since even the most extensive genomes still offer an adequate selection of sequences that are not present. In the event that the sequence that is not present differs from a sequence that is present by only one base, the hybridization step must be carried out under strict conditions. Preferably, however, sequences are used that differ significantly, i.e. in multiple bases, from the sequences that are present in the sample. Particularly good results are achieved when oligonucleotides having an identical or at least a similar number of bases are used for the test sites and for the reference sites.

The ligate nucleic acid oligomers of the present invention consist of n nucleotides, which are present in a specific nucleotide sequence. The ligate nucleic acid oligomers are complementary, or at least predominantly complementary, to a sequence region of n nucleotides of the nucleic acid oligomer ligands. The nucleic acid oligomer ligands whose presence is to be detected based on the present invention may exhibit further sequence regions in addition to the n-nucleotide-long contiguous sequence region whose sequence is complementary to the n-nucleotide sequence of the ligates.

According to a preferred embodiment of the present invention, ligate nucleic acid oligomers are used, one type of ligate nucleic acid oligomer consisting each time of a number of n nucleotides having a specific sequence. The ligate nucleic acid oligomers of one type are complementary to a sequence region having n nucleotides of nucleic acid oligomer ligands of one type, the ligate nucleic acid oligomer being able to form a perfect match with this n-nucleotide-long contiguous sequence region of the nucleic acid oligomer ligand, i.e. in this n-nucleotide-long region, following association (hybridization), ligate and matching ligand form a double helix having n exclusively matching base pairings. The signal nucleic acid oligomer ligands that associate to this type of ligate nucleic acid oligomer exhibit, in contrast, sequence regions that are only partially complementary to the n-nucleotide-long sequence of the ligates, in particular, the signal nucleic acid oligomer ligands possess only such sequence sections that, following association with the n-nucleotide-long sequence of the ligate nucleic acid oligomer, a maximum of n-1, n-2, n-3, n4 or n-5 nucleotides are complementary to each other. In this context, n represents an integer from 3 to 80, especially from 5 to 50, particularly preferably from 15 to 35 or from 8 to 25. In the following, signal nucleic acid oligomer ligands that satisfy these conditions will also be called SNP-ID ligands.

According to a particularly preferred embodiment, the signal nucleic acid oligomer ligands that are used as SNP-ID ligands consist of fewer than n nucleotides, all nucleotides of the signal nucleic acid oligomer ligand being sequence-specifically complementary to the n-nucleotide sequence of the ligate nucleic acid oligomer. Alternatively, also signal nucleic acid oligomer ligands may be used that consist of n or more nucleotides but exhibit only regions comprising n nucleotides whose sequence is complementary to the sequence of the n-nucleotide-long ligate nucleic acid oligomer in fewer than n nucleotides, especially in n-1 to n-5 nucleotides. Alternatively, also signal nucleic acid oligomer ligands may be used that consist of n or more nucleotides but exhibit only such partial sequences that, following association of two or more partial sequences of the signal nucleic acid oligomer ligand to the ligate nucleic acid oligomer, fewer than n nucleotides of the hybrid comprising ligate and signal nucleic acid oligomer ligand, especially only n-1 to n-5 nucleotides, may be present complementary to each other in hybridized form.

These coordinated complementary regions of ligate nucleic acid oligomers and signal nucleic acid oligomer ligands entail a particular advantage for the detection of nucleic acid oligomer hybridization events. In principle, in the methods according to the present invention, only those hybridization events in which a nucleic acid oligomer ligand hybridizes to the ligate nucleic acid oligomer are to be detected, the nucleic acid oligomer ligand exhibiting a contiguous nucleotide sequence that is complementary to the ligate nucleic acid oligomer in all bases, thus allowing a so-called perfect match to form. In order to discriminate between such perfect congruences and associates with base mismatches, i.e. to suppress or detect as negative hybridization events in which the associate (hybrid) comprising ligate nucleic acid oligomer and nucleic acid oligomer ligand exhibits one or more base pairs that are not complementary to each other, the association constants (and times) of perfect match, K_{M}, mismatch, K_{MM}, and hybrid comprising ligate nucleic acid oligomer and signal nucleic acid oligomer ligand, K_{SIG}, are crucial. The association constant K_{SIG} of the hybrid comprising ligate nucleic acid oligomer and signal nucleic acid oligomer ligand must namely be greater than the association constant K_{MM} of a mismatch, since in this case the nucleic acid oligomer ligands whose association exhibits at least one mismatch with the ligate nucleic acid oligomers can be displaced or largely displaced from their bond to the ligate nucleic acid oligomers by the signal nucleic acid oligomer ligands and, conversely, signal nucleic acid oligomers that are already associated to the ligate nucleic acid oligomer can no longer be displaced from the associate by mismatch nucleic acid oligomer ligands, or only very little. Simultaneously, however, the association constant K_{SIG} of the hybrid comprising ligate nucleic acid oligomer and signal nucleic acid oligomer ligand must be less than the association constant K_{M} of a perfect match, since in this case the nucleic acid oligomer ligands whose association with the ligate nucleic acid oligomers form a perfect match cannot be displaced from their bond to the ligate nucleic acid oligomers by the signal nucleic acid oligomer ligands and, conversely, signal nucleic acid oligomers that are already associated to the ligate nucleic acid oligomer are displaced from the associate by a nucleic acid oligomer ligand that allows a perfect match. Signal nucleic acid oligomer ligands that satisfy these conditions are referred to as SNP-ID ligands. These SNP-ID ligands possess only such sequence sections that, following association with the ligate nucleic acid oligomer, a maximum of n-1, n-2, n-3, n-4 or n-5 nucleotides are complementary to each other. The correlation K_{M}>K_{SIG}≧K_{MM} then applies to the association constants of the possible hybrids. Given suitable selection of the signal nucleic acid oligomer ligand, especially if signal nucleic acid oligomer ligands are used that consist of a total of fewer than n nucleotides, all nucleotides of the signal nucleic acid oligomer ligand being sequence-specifically complementary to the n-nucleotide sequence of the ligate nucleic acid oligomer, the association constant of the hybrid comprising ligate nucleic acid oligomer and signal nucleic acid oligomer ligand is somewhat greater than the association constant of a mismatch, since the non-complementary bases of the mismatch are usually located in the interior of the nucleic acid chain. The hybrid is damaged more severely by this than by a missing base on the chain end, making the association constant of the hybrid comprising ligate nucleic acid oligomer and shortened signal nucleic acid oligomer ligand somewhat greater than the association constant of a mismatch, and a correlation K_{M}>K_{SIG}≧K_{MM} can thus be set.

In this context, it should be made clear that those of ordinary skill in the art are aware that differences in the association behavior of differing ligate/ligand pairs resulting from different association constants can be increased or compensated for by varying the concentrations of ligates and ligands provided for association or that, if differing ligate/ligand pairs have similar association constants, differences in the association behavior of differing ligate/ligand pairs can be produced by varying the concentrations of ligates and ligands provided for association.

The above-described coordinated lengths of ligate nucleic acid oligomers and signal nucleic acid oligomer ligands entail a most particular advantage for detection of SNPs (single nucleotide polymorphisms). SNPs, namely, exhibit a mutation in precisely one base, which can be unambiguously discriminated from the non-mutated variant by the described method.

Here, it should be mentioned that the considerations regarding the association constants when using ligate nucleic acid oligomers and signal nucleic acid oligomer ligands with coordinated lengths can be carried over to displacement assays using e.g. ligate antibodies, signal-antigen ligands and antigen ligands. In place of the nucleotide regions for sequence-specific hybridization and the association constants K_{M}, K_{SIG} and K_{MM} associated therewith, in this case the association constants of the signal-antigen/ligate-antibody and antigen-ligand/ligate-antibody associates differ e.g. by an order of magnitude. Essentially, therefore, in the above statements, K_{M}, K_{SIG} and K_{MM} are replaced by A_{M} and A_{SIG}, A_{SIG} indicating the association constant of the signal-antigen/ligate-antibody associates and A_{M} the association constants of the antigen-ligand/ligate-antibody associates, and e.g. A_{M} is greater than A_{SIG} by an order of magnitude, i.e. A_{M}=10×A_{SIG}.

For the use of ligate nucleic acid oligomers and signal nucleic acid oligomer ligands with coordinated lengths just described, it should be kept in mind that the association constant of the hybrid comprising ligate nucleic acid oligomer and signal nucleic acid oligomer ligand and the association constant of a perfect match exhibit a very similar value. For this reason, the following embodiments (i) to (iii) are particularly preferred:

(i) If, first, the nucleic acid oligomer ligands are added and—after removing unassociated nucleic acid oligomer ligands by washing the modified surface—separately, the signal nucleic acid oligomer ligands are added, a molar ratio of signal nucleic acid oligomer ligands to ligate nucleic acid oligomers between 0.01 and 1000, preferably between 0.1 and 100, particularly preferably between 1 and 10 is used. In this way, displacement of the nucleic acid oligomers from the perfect matches by the signal nucleic acid oligomer ligands is avoided.

(ii) If nucleic acid oligomer ligands and signal nucleic acid oligomer ligands are jointly brought into contact with the modified surface, a molar ratio of signal nucleic acid oligomer ligands to nucleic acid oligomer ligands between 0.01 and 1000, preferably between 0.1 and 100, particularly preferably between 1 and 10 is used. The expected quantity of the nucleic acid oligomer ligands must be known precisely to at least one order of magnitude. If it is not known, however, a series of experiments can be carried out with varying molar ratios of signal nucleic acid oligomer ligands to ligate nucleic acid oligomers in the range between 0.01 and 1000.

(iii) If, first, the signal nucleic acid oligomer ligands are added and—after removing unassociated signal nucleic acid oligomer ligands by washing the modified surface—separately, the nucleic acid oligomer ligands are added, in order to allow gradual displacement of the signal nucleic acid oligomer ligands from the associate with the ligate nucleic acid oligomers by perfectly hybridizing nucleic acid oligomer ligands, and to discriminate these from displacement of the signal nucleic acid oligomer ligands from the associate with the ligate nucleic acid oligomers by mismatch-forming nucleic acid oligomer ligands, a titration curve is recorded, i.e. the sample is added gradually in small quantities and, after each addition, a measured value is recorded. The resultant measured value curve, comprising measured values versus added sample portions, then exhibits a significant increase in measured value when few sample portions are added, if perfectly hybridizing nucleic acid oligomer ligands were present in the sample, while nucleic acid oligomer ligands that form mismatches during hybridization lead to an increase in the measured values only when significantly more sample is added, and these two measured value ranges—given the presence of matches and mismatches in the sample—are separated from one another by a plateau in the measured values.

According to a particularly preferred embodiment of the present invention, when SNP-ID ligands are used as signal nucleic acid oligomers, the modified surface is washed before the first detection of the signal nucleic acid oligomer ligands. In this way, the excess solution with the SNP-ID ligands contained therein that are not associated to ligate nucleic acid oligomers are removed. During the subsequent detection of the signal nucleic acid oligomer ligands, a signal is measured that originates exclusively from the SNP-ID ligands bound to the ligate nucleic acid oligomers. The superpositioning of the measured values by unbound signal nucleic acid oligomer ligands is thus prevented, achieving a higher-precision detection.

In the event that at least two types of ligate nucleic acid oligomers are attached to the modified surface, according to a preferred embodiment of the present invention, if SNP-ID ligands are used as signal nucleic acid oligomers, also such ligate nucleic acid oligomers are used in which the differing types of ligate nucleic acid oligomers, especially those in the measurement region M, each exhibit a different number n_{Mi} of nucleotides, n_{Mi} representing an integer and indicating the number of nucleotides of one type of ligate nucleic acid oligomer that was immobilized on the test site i in the measurement region M. In this case, the type of SNP-ID ligand that matches each type of ligate nucleic acid oligomer is adapted, as mentioned above, such that it exhibits only those sequence sections that, following association with the n_{Mi}-nucleotide-long sequence of the ligate nucleic acid oligomer in the hybrid, a maximum of n_{Mi}-1, n_{Mi}-2, n_{Mi}-3, n_{Mi}-4 or n_{Mi}-5 exhibit nucleotides that are complementary to each other. The variation of the nucleotide length of the ligate nucleic acid oligomers in parallel detection of differing nucleic acid oligomer ligands allows a more targeted and simpler selection of ligate nucleic acid oligomers that permit a perfect match with the nucleic acid oligomer ligands when the nucleic acid oligomer ligands of the sample to be analyzed constitute a large pool of potentially existing nucleic acid oligomer ligands, as is the case e.g. for SNP analyses of the entire human genome. If SNP-ID ligands are used, it is namely no longer necessary to take care that the stringent hybridization conditions usually needed for SNP analyses in order to discriminate matches from mismatches are set to be identical for all test sites in the parallel analysis on a surface. In fact, the adjusted SNP-ID ligands allow indirect stringent hybridization that is carried out simultaneously for all test sites but is individually adapted to each test site.

According to a further preferred embodiment of the present invention, ligate PNA oligomers are used as ligate nucleic acid oligomers and signal PNA oligomer ligands are used as signal nucleic acid oligomer ligands. The advantage of using ligate PNA oligomers and signal PNA oligomer ligands is grounded in the fact that the methods of the present invention can be carried out with no additional modification of ligate nucleic acid oligomers, nucleic acid oligomer ligands or signal nucleic acid oligomer ligand with detectable label. As is known, PNA differs from DNA in its lack of electrical charge. If ligate PNA oligomers and signal PNA oligomer ligands are used, the charge of the nucleic acid oligomer ligand can be regarded as a “label” that can be detected by suitable measurements, especially by electrochemical measurements such as electrochemical impedance measurements or by SECM.

In addition, the present invention is also directed to a kit for carrying out a method for detection of nucleic acid oligomer hybridization events. The kit comprises a modified surface, the modification consisting in the attachment of at least one type of ligate nucleic acid oligomer; and an effective quantity of signal nucleic acid oligomer ligands. With this kit, a two-fold detection of the signal nucleic acid oligomer ligands can be carried out, i.e. a determination of the reference values and the second detection of the signal nucleic acid oligomer ligands, as well as a measurement of correction values.

According to a preferred embodiment, the reference values are already comprised by the kit, so that the signal nucleic acid oligomer ligands must be detected by the end consumer only once. The values obtained from this detection then need only be compared with the already existing reference values.

According to a preferred embodiment, the reference values and correction values are already comprised by the kit, so that the signal nucleic acid oligomer ligands must be detected by the end consumer only once. The values obtained from this detection then need only be compared with the already existing reference values and standardized to the correction values.

According to a preferred embodiment of the present invention, the kit comprises a modified surface that exhibits at least one T₀ region and at least one T₁₀₀ region. Embodiments according to which the modified surface additionally comprises at least one Tₙ region are particularly preferred.

In addition, the present invention is also directed to a kit for carrying out a method for detection of SNP analyses. The kit comprises a modified surface, the modification consisting in the attachment of at least one type of ligate nucleic acid oligomer; and an effective quantity of SNP-ID ligands. With this kit, a two-fold detection of the SNP-ID ligands can be carried out, i.e. a determination of the reference values and the second detection of the signal nucleic acid oligomer ligands, as well as a measurement of correction values.

According to a preferred embodiment, the reference values and correction values are already comprised by the kit, so that the SNP-ID ligands must be detected by the end consumer only once. The values obtained from this detection then need only be compared with the already existing threshold values determined from reference values and correction values to recognize the absence or presence of an SNP and its composition (homozygote or heterozygote). The present invention thus provides a method for detection of sequence-specific nucleic acid oligomer hybridization events based on a displacement assay. In doing so, ligate nucleic acid oligomers such as DNA/RNA/PNA oligomer single-strands immobilized on surfaces serve as an association matrix (probe) for detection of targets, i.e. for detection of oligonucleotides or DNA fragments. First, the ligate nucleic acid oligomers of the association matrix are brought into contact with a solution of signal nucleic acid oligomer ligands, causing some of the signal nucleic acid oligomer ligands to be hybridized to the surface-immobilized ligate nucleic acid oligomers and the remaining signal nucleic acid oligomer ligands to remain in the excess solution. Alternatively, if SNP-ID ligands are used as signal nucleic acid oligomers, before the signal nucleic acid oligomer ligands are detected, the modified surface may be washed. In this way, the excess solution with the SNP-ID ligands contained therein that are not associated to ligate nucleic acid oligomers is removed. The signal nucleic acid oligomer ligands are chosen such that the surface associates comprising ligate nucleic acid oligomer and signal nucleic acid oligomer ligand possess an association constant that is less than the association constant between ligate nucleic acid oligomer and nucleic acid oligomer ligand. In addition, the signal nucleic acid oligomer ligands or the nucleic acid oligomer ligands either function themselves as the signal-generating substance for detection, or they are labeled with a detectable signal-generating substance. In a (reference) detection, the surface-immobilized signal nucleic acid oligomer ligands are captured in the absence of nucleic acid oligomer ligands—either before the addition of the ligand nucleic acid oligomers or after dissociation of the ligand oligonucleotides—using a suitable measuring method. If signal nucleic acid oligomer ligands are located in the excess solution during the reference measurement, a suitable surface-sensitive measuring method (e.g. total internal reflection fluorescence or electrochemical methods such as chronocoulometry) that permits discriminating between surface-immobilized signal nucleic acid oligomer ligands and signal nucleic acid oligomer ligands in the volume phase is chosen as the measuring method. In a further measurement—after addition to the modified surface of the solution containing nucleic acid oligomer ligand to be analyzed and displacement of a portion of the signal nucleic acid oligomer ligands by the ligate nucleic acid oligomers, or after the addition of the ligand oligonucleotides, (stringent) hybridization, washing and addition of an identical quantity of signal oligonucleotides as for the reference measurement—the reference signal present in the ligate nucleic acid oligomer/signal nucleic acid oligomer ligand system is modulated, thus allowing qualitative and quantitative statements about nucleic acid oligomer ligands in the test solution. The association matrix may consist of only one test site with one type of ligate nucleic acid oligomer, but it is preferred that the association matrix consist of a plurality of test sites.

Thus, in principle, the determination of an unknown type of nucleic acid oligomer ligand takes place by detecting a third link (signal nucleic acid oligomer ligand) that, like the unknown type of nucleic acid oligomer ligand, associates to a probe molecule (ligate nucleic acid oligomer). If signal nucleic acid oligomer ligand and nucleic acid oligomer ligand are present, due to the stronger association ability of the unknown substance (nucleic acid oligomer ligand), at least a portion of the ligate nucleic acid oligomer is coated. Likewise, of course, if associations comprising nucleic acid oligomer ligand and ligate nucleic acid oligomer are present, a portion of the nucleic acid oligomer ligands may be displaced from the ligand-ligate complex by adding signal nucleic acid oligomer ligands. This occurs to a small degree despite the stronger association ability of nucleic acid oligomer ligand with ligate nucleic acid oligomer since, in any case, a balance will be reached that is determined by the ratio of the association constants of nucleic acid oligomer ligand/ligate nucleic acid oligomer to signal nucleic acid oligomer ligand/ligate nucleic acid oligomer and the concentrations of nucleic acid oligomer ligand and signal nucleic acid oligomer ligand used.

The displacement assay thus comprises a complexation event between a ligate nucleic acid oligomer and a signal nucleic acid oligomer ligand that competes with a further complexation event comprising actual target (nucleic acid oligomer ligand) and ligate nucleic acid oligomer. The displacement assay especially comprises a complexation event between a ligate nucleic acid oligomer and a signal nucleic acid oligomer ligand, which is joined by a further complexation event after the actual target (nucleic acid oligomer ligand) is added, which takes place and displaces the signal nucleic acid oligomer ligand. In addition, the displacement assay especially comprises a complexation event between a ligate nucleic acid oligomer and a target (nucleic acid oligomer ligand), which is joined by a further complexation event after signal nucleic acid oligomer ligands are added, which may take place and displace at least a portion of the nucleic acid oligomer ligands.

The Surface

The term “surface” refers to any support material that is suitable for binding derivatized or non-derivatized ligate nucleic acid oligomers covalently or via other specific interactions, directly or following appropriate chemical modification. The solid support may consist of conductive or non-conductive material.

(i) Conductive Surfaces

The term “conductive surface” is understood to mean any support having an electrically conductive surface of any thickness, especially surfaces comprising platinum, palladium, gold, cadmium, mercury, nickel, zinc, carbon, silver, copper, iron, lead, aluminum and manganese.

In addition, any doped or undoped semiconductor surfaces of any thickness may also be used. All semiconductors may be used in the form of pure substances or in the form of composites. Examples include, but are not limited to, carbon, silicon, germanium, α tin, and Cu(I) and Ag(I) halides of any crystal structure. All binary compounds of any composition and any structure comprising the elements of groups 14 and 16, the elements of groups 13 and 15, and the elements of groups 15 and 16 are also suitable. In addition, ternary compounds of any composition and any structure comprising the elements of groups 11, 13 and 16 or the elements of groups 12, 13 and 16 may be used. The designations of the groups of the periodic table of the elements refer to the IUPAC recommendation of 1985.

(ii) Non-Conductive Surfaces

The material preferred for non-conductive surfaces is glass and modified glass. The modification may take place e.g. by silanization and, in all cases, results in functional groups that are suitable for binding, in coupling reactions, appropriately functionalized ligate nucleic acid oligomers. This modification includes layered superstructures on the glass surface when using polymers, such as dextran polymers, that allow a variation of the layer thickness and surface condition. Further derivatization options of the glass for the ultimate attachment of the ligate nucleic acid oligomers consist, e.g., in applying a thin (approximately 10-200 nm) metallization layer, especially a gold metallization layer, which may additionally be coated with (thiol-functionalized) polymers, especially dextrans. In addition, following silanization, the glass may also be functionalized with biotin (e.g. amino-functionalized glass surface following silanization and coupling of the carboxylic acid biotin via EDC and NHS or via a biotin active ester such as biotin-N-succinimidyl ester) or, alternatively, coated with a dextran lysine or dextran-immobilized biotin. Thereafter, the biotinylated glass surfaces produced in this way are treated with avidin or streptavidin and may then be used for the attachment of biotinylated ligate nucleic acid oligomers.

Binding Nucleic Acid Oligomers to the Surface

In the context of the present invention, nucleic acid oligomers bound to a surface are referred to as ligate nucleic acid oligomers. Methods for immobilization of nucleic acid oligomers to a surface are known to those of ordinary skill in the art. The ligate nucleic acid oligomers may, e.g., be covalently bound to the surface via hydroxyl, epoxide, amino or carboxy groups of the support material having thiol, hydroxy, amino or carboxyl groups that are naturally present on the nucleic acid oligomer or that have been affixed to the ligate nucleic acid oligomer by derivatization. The ligate nucleic acid oligomer may be bound to the surface atoms or molecules of a surface directly or via a linker/spacer. In addition, the ligate nucleic acid oligomer may be anchored by the methods common in immunoassays, such as by using biotinylated ligate nucleic acid oligomers for non-covalent immobilization to avidin or streptavidin-modified surfaces. The chemical modification of the ligate nucleic acid oligomers with a surface anchor group may be introduced as early as in the course of automated solid-phase synthesis, or in separate reaction steps. In this process, the nucleic acid oligomer is also linked directly or via a linker/spacer with the surface atoms or surface molecules of a surface of the type described above. This bond may be accomplished in various ways (cf. e.g. WO 00/42217).

Ligate Nucleic Acid Oligomers/Nucleic Acid Oligomer Ligands/Signal Nucleic Acid Oligomer Ligands

The ligate nucleic acid oligomers of the present invention consist of n nucleotides, which are present in a specific nucleotide sequence. The ligate nucleic acid oligomers are complementary, or at least predominantly complementary, to a sequence region of n nucleotides of the nucleic acid oligomer ligands.

Molecules that specifically interact with the ligate nucleic acid oligomers that are immobilized to a surface, forming a double-strand hybrid, are referred to as nucleic acid oligomer ligands. Nucleic acid oligomer ligands within the meaning of the present invention are thus nucleic acid oligomers that function as complex binding partners of the complementary nucleic acid oligomer. The nucleic acid oligomer ligands whose presence is to be detected based on the present invention may exhibit further sequence regions in addition to the n-nucleotide-long contiguous sequence region whose sequence is complementary to the n-nucleotide sequence of the ligates.

The ligate nucleic acid oligomers of one type are complementary to an n-nucleotide sequence region of nucleic acid oligomer ligands of one type, the ligate nucleic acid oligomer being able to form a perfect match with this n-nucleotide-long contiguous sequence region of the nucleic acid oligomer ligand, i.e. in this n-nucleotide-long region, after association (hybridization), ligate and matching ligand form a double helix having n exclusively matching base pairings.

In contrast, the signal nucleic acid oligomer ligands that associate to one type of ligate nucleic acid oligomer exhibit sequence regions that are only partially complementary to the n-nucleotide-long sequence of the ligate.

In a particular embodiment of the present invention, the signal nucleic acid oligomer ligands possess only such sequence sections that, following association with the n-nucleotide-long sequence of the ligate nucleic acid oligomer, a maximum of n-1, n-2, n-3, n4 or n-5 nucleotides are complementary to each other. In this context, n represents an integer from 3 to 80, especially from 5 to 50, particularly preferably from 15 to 35 or from 8 to 25. Signal nucleic acid oligomer ligands that satisfy these conditions are also called SNP-ID ligands. The signal nucleic acid oligomer ligands can, themselves or following appropriate modification with a detection label, be determined by suitable detection methods. The detection of the signal nucleic acid oligomer ligands may take place by a surface-sensitive detection method, especially by a spectroscopic, an electrochemical or an electrochemiluminescent method.

As spectroscopic detection, detection of the fluorescence, especially the total internal reflection fluorescence (TIRF), of the signal nucleic acid oligomer ligands may be considered, whereas electrochemical detection may take place by amperometry, chronocoulometry, impedance measurement or scanning electrochemical microscopy (SECM).

In an alternative embodiment of the present invention, ligate PNA oligomers are used as ligate nucleic acid oligomers, and signal PNA oligomer ligands are used as signal nucleic acid oligomer ligands. The advantage of using ligate PNA oligomers and signal PNA oligomer ligands is grounded in the fact that any embodiment of the displacement assay of the present invention may be carried out with no additional modification of ligate nucleic acid oligomer, nucleic acid oligomer ligand or signal nucleic acid oligomer ligand with a detectable label. As is known, PNA differs from DNA in its lack of electrical charge. If ligate PNA oligomers and signal PNA oligomer ligands are used, the charge of the nucleic acid oligomer ligand can be regarded as a “label” that can be detected by suitable measurements, especially by electrochemical measurements such as electrochemical impedance measurements or by SECM.

In a further alternative embodiment, SNP-ID ligands are used as signal nucleic acid oligomer ligands. According to a particularly preferred embodiment, before the signal nucleic acid oligomer ligands are detected, the modified surface is washed. In this way, the excess solution with the SNP-ID ligands contained therein that are not associated to ligate nucleic acid oligomers is removed. In the subsequent detection of the signal nucleic acid oligomer ligands, a signal is measured that originates exclusively from the SNP-ID ligands bound to the ligate nucleic acid oligomers. The superpositioning of the measured values by unbound signal nucleic acid oligomer ligands is thus prevented, achieving a detection with higher precision and allowing any method that is sensitive to the relevant detection label used to be used as the measuring method.

In the context of the present invention, the signal nucleic acid oligomer ligands possess a lower tendency to form complexes with the ligate nucleic acid oligomers than do the actual nucleic acid oligomer ligands (targets), i.e. the association constant between ligand and ligate is greater than the association constant between signal ligand and ligate.

Thus, in the case of a ligate oligonucleotide with 20 bases, the signal oligonucleotide exhibits only nucleic acid regions in which fewer than 20 consecutive bases of the signal oligonucleotide are complementary to the 20 bases of the ligate oligonucleotide. Thus, for a ligate oligonucleotide with 20 nucleotides, a detection-labeled signal oligonucleotide of any length may be used as long as the signal oligonucleotide exhibits only nucleic acid regions in which fewer than 20 consecutive bases of the signal oligonucleotide are complementary to the 20 bases of the ligate oligonucleotide. In particular, the signal oligonucleotide may be a 20-nt oligo labeled with at least one detection label, or contain a 20-nt oligo sequence that is complementary to the 20-nt ligate oligonucleotide and that forms one or more base-pair mismatches during complexation between signal oligonucleotide and ligate oligonucleotide. An oligonucleotide that exhibits fewer than 20 nt and that is completely complementary to the ligate oligonucleotide and labeled with at least one detection label may also be used.

In the context of the present invention, a compound comprising at least two covalently-joined nucleotides or at least two covalently-joined pyrimidine (e.g. cytosine, thymine or uracil) or purine bases (e.g. adenine or guanine), preferably a DNA, RNA or PNA fragment, is used as the nucleic acid oligomer. The term nucleic acid refers to any backbone of the covalently-joined pyrimidine or purine bases, such as the sugar-phosphate backbone of DNA, cDNA or RNA, a peptide backbone of PNA, or analogous backbone structures, such as a thiophosphate, a dithiophosphate or a phosphoramide backbone. An essential feature of a nucleic acid within the meaning of the present invention is the sequence-specific binding of naturally occurring DNA or RNA or structures derived (transcribed or amplified) therefrom, such as cDNA or amplified cDNA or amplified RNA (aRNA).

Detection Label/Marker (Marker Molecule)

Signal nucleic acid oligomer ligands that cannot be detected themselves are provided with one or more detectable labels by derivatization. This label allows detection of the complexation events between the signal nucleic acid oligomer ligand and the surface-bound ligate nucleic acid oligomer. The label can supply a detection signal directly or, as in the case of enzyme-catalyzed reactions, indirectly. Preferred detection labels (marker molecules) are fluorophores and redox-active substances.

If ligate PNA oligomers are used as ligate nucleic acid oligomers and signal PNA oligomer ligands are used as signal nucleic acid oligomer ligands, work can be done with no additional modification of ligate nucleic acid oligomer, nucleic acid oligomer ligand or signal nucleic acid oligomer ligand with detectable labels. In this case, the charge of the nucleic acid oligomer ligand is used as the “label”, which can be detected by suitable measurements, especially by electrochemical measurements such as electrochemical impedance measurements or by SECM. For the fluorophores, commercially available fluorescent dyes such as Texas Red, rhodamine dyes, fluorescein, etc. are used (cf. Molecular Probes Catalog). In the event of detection by electrochemical methods, redox molecules are employed as labels. Transition metal complexes, especially those of copper, iron, ruthenium, osmium or titan may be used as redox labels with ligands such as pyridine, 4,7-dimethylphenanthroline, 9,10-phenanthrene quinonediimine, porphyrins and substituted porphyrin derivatives. In addition, it is possible to employ riboflavin, quinones such as pyrrolloquinoline quinone, ubiquinone, anthraquinone, naphthoquinone or menaquinone, or derivatives thereof, metallocenes and metallocene derivatives such as ferrocenes and ferrocene derivatives, cobaltocenes and cobaltocene derivatives, porphyrins, methylene blue, daunomycin, dopamine derivatives, hydroquinone derivatives (para- or ortho-dihydroxybenzene derivatives, para- or ortho-dihydroxyanthraquinone derivatives, para- or ortho-dihydroxynaphthoquinone derivatives) and similar compounds.

Surface-Sensitive Detection Methods

Surface-sensitive detection methods allow discrimination between marker molecules associated to a surface and those that are dissolved in excess. Electrochemical, spectroscopic and electrochemiluminescent methods are suitable as the detection method.

(i) Surface-Sensitive Electrochemical Detection

In electrochemical methods, in principle, the kinetics of the electrochemical processes may be used to discriminate between redox-active detection labels adsorbed to a surface and those dissolved in excess. Generally, surface-adsorbed detection labels are electrochemically converted (e.g. oxidized or reduced) more quickly than redox-active detection labels from the volume phase, since the latter must first diffuse to the (electrode) surface before electrochemical conversion. Examples of electrochemical surface-sensitive methods include amperometry and chronocoulometry.

The chronocoulometry method allows discriminating surface-near redox-active components from (identical) redox-active components in the volume phase and is described, for example, in Steel, A. B., Herne, T. M. and Tarlov M. J.: Electrochemical Quantitation of DNA Immobilized on Gold, Analytical Chemistry, 1998, Vol. 70, 4670-4677 and the literature cited therein.

The chronocoulometry measurement signal (transferred charge Q as a function of time t) is composed of three components: (i) a diffusive portion, which is brought about by the dissolved redox-active components in the volume phase and exhibits a t^{1/2} dependence, a first instantaneous portion, which results from the charge redistribution in the double layer (dl) on the electrode surface, and a second instantaneous portion, which is caused by the conversion of redox-active components adsorbed (immobilized) on the electrode surface.

In the chronocoulometric experiment, the Cottrell equation gives the charge Q as a function of time t:

$$Q = \frac{2 {nFAD}_{0}^{1 / 2} {C}_{0}^{*}}{{π}^{1 / 2}} {t}^{1 / 2} + {Q}_{dl} + nFA {Γ}_{0}$$

wherein
n: number of electrons per molecule for the reduction
F: Faraday constant
A: electrode surface [cm²]
D₀: diffusion coefficient [cm²/s]
C₀*: concentration [mol/cm²]
Q_{dl}: capacitive charge C
nFAΓ₀: charges that are converted during the electrochemical conversion of the adsorbed redox-active detection label, wherein Γ₀ [mol/cm²] represents the surface concentration of the detection label.

The term Γ₀ thus represents the quantity of detection label on the electrode surface. In the chronocoulometric experiment, when t=0, the sum of the double-layer charges and the surface excess is maintained.

A chronocoulometrically detected displacement assay within the meaning of the present invention will be explained using the example of a 20-nt ligate nucleic acid oligomer. The (working) electrode modified with 20-nt ligate oligonucleotides is brought into contact with a defined quantity of signal nucleic acid oligomer ligands, e.g. a 12-nt signal nucleic acid oligomer ligand that carries one or more redox labels and is complementary to a region of the ligate oligonucleotide that is as close to the surface as possible so that association can occur between ligate oligonucleotide and redox-labeled ss nucleic acid oligomer complex former. Thereafter, the (working) electrode is initially set to a potential E₁ at which little to no electrolysis (electrochemical change in the redox state) of the redox label can occur (e.g., for ferrocene-modified ligate oligonucleotide, approximately 0.1 V versus Ag/AgCl (sat. KCl)). Then the working electrode is set by a potential jump to a potential E₂ at which the electrolysis of the redox label occurs in the diffusion-limited borderline case (e.g., for ferrocene-modified ss nucleic acid oligomer complex former, approximately 0.5 V versus Ag/AgCl (sat. KCl)). The transferred charges are recorded as a function of time.

Thereafter, the sample solution is added that is supposed to (may) contain the ligand nucleic acid oligomer (target) that exhibits an nt sequence that, in one region, is complementary to the 20 nt of the ligate oligonucleotides. Following hybridization of the target to the ligate oligonucleotides, and thus following partial displacement of the signal nucleic acid oligomer ligands, a second electrochemical measurement is carried out. The change in the instantaneous charge signal is proportional to the number of displaced signal oligonucleotide ligands and is thus proportional to the number of target oligonucleotides present in the test solution.

This change in the instantaneous charge signal depends on the length of the ligand oligonucleotides, that is, on the number of nucleotides of the ligand oligonucleotides. If the ligand oligonucleotides exhibit a length that approximately corresponds to or is shorter than the length of the ligate oligonucleotides, a decrease in the instantaneous charge signal will be observed, since signal oligonucleotides are displaced by the ligand oligonucleotides from the associate with the ligate oligonucleotides, and thus from the proximity of the modified surface. If the ligand oligonucleotides exhibit a longer length than the ligate oligonucleotides, only a portion of the nucleotides of the ligand oligonucleotides can attach to the nucleotides of the ligate oligonucleotides and an excess portion of the ligand oligonucleotide with freely accessible nucleotides remains. Generally, following hybridization of the ligand oligonucleotides to the ligate oligonucleotides, the signal oligonucleotides then attach to these free bases of the ligand oligonucleotides. If the excess portion of the ligand oligonucleotides is located near the surface, it may happen that an increase in the instantaneous charge signal is observed, since the number of signal oligonucleotides located near the surface increases through the addition of the long-chain ligand oligonucleotides and binding of the signal oligonucleotides to these ligand oligonucleotides. For very long-chain ligand oligonucleotides, a considerably greater difference (increase) may result between the instantaneous charge signal before the addition of ligand oligonucleotide and the instantaneous charge signal after the addition of ligand oligonucleotide than for short-chain ligand oligonucleotides (decrease in the instantaneous charge signal). Uncertain results are obtained only when the number of signal nucleic acid oligomer ligands that are displaced from the signal ligand oligonucleotide/ligate oligonucleotide associate approximately corresponds to the number of signal nucleic acid oligomer ligands that subsequently attach to the portion of the ligand oligonucleotides that are located near the surface and that exceeds the portion associated to the ligate oligonucleotide.

(ii) Surface-Sensitive Fluorescence Detection

Total internal reflection fluorescence (TIRF, cf. Sutherland and Dahne, 1987, J. Immunol. Meth., 74, 253-265) can serve as an optical measuring method for detection of fluorescence-labeled signal nucleic acid oligomer ligands. Here, fluorescence molecules that are located near the interface between a solid waveguide medium, typically glass, and a liquid medium, or that are immobilized on the waveguide medium surface facing the liquid, can be excited by the evanescent field protruding from the waveguide and emit detectable fluorescence light. Fluorescence-labeled complex formers that are displaced or dissolved in the excess are not captured by the evanescent field (or only to the extent that they are located in the range of the penetration depth of the evanescent field) and thus contribute (nearly) nothing to the measured signal. The penetration depth of the evanescent field is typically 100 to 200 nm, but it may also be increased by a thin metallization layer (approximately 10 to 200 nm), especially a gold metallization layer, to several 100 nm. In a preferred embodiment of the fluorescence detection of the displaced, fluorophore-labeled signal nucleic acid oligomer ligands, the layer thickness of the ligate-modified support surface is adjusted to the penetration depth of the evanescent field, e.g.
by appropriately long ligate nucleic acid oligomers,
by immobilizing the ligate nucleic acid oligomers via appropriately long linkers between surface and ligate oligonucleotide,
by coupling the carboxylic acid biotin (via EDC and NHS or via a biotin active ester such as biotin-N-succinimidyl ester) to amino-derivatized surfaces and coupling avidin or streptavidin to the biotinylated surfaces produced in this way, with subsequent attachment of biotinylated ligate nucleic acid oligomers or
by immobilizing an appropriately thick layer on functionalized polymer and attaching the ligate nucleic acid oligomer to the polymer, e.g. (a) by applying a thin (approximately 10-200 nm) metallization layer, especially a gold metallization layer, which may be coated with a (thiol-functionalized) polymer, especially dextrans or polylysine, which, in turn, is used for the attachment of the ligate nucleic acid oligomer, or (b) by applying a polymer layer comprising polylysine-biotin, dextran-lysine-biotin or dextran-immobilized biotin and coupling avidin or streptavidin to the biotinylated surfaces produced in this way, with subsequent attachment of biotinylated ligate nucleic acid oligomers.

Non-Surface-Sensitive Detection Methods

In a further alternative embodiment, SNP-ID ligands are used as signal nucleic acid oligomer ligands. According to a particularly preferred embodiment, before the signal nucleic acid oligomer ligands are detected, the modified surface is washed. In this way, the excess solution with the SNP-ID ligands contained therein that are not associated to ligate nucleic acid oligomers is removed. During the subsequent detection of the signal nucleic acid oligomer ligands, a signal is measured that originates exclusively from the SNP-ID ligands bound to the ligate nucleic acid oligomers. The superpositioning of the measured values by unbound signal nucleic acid oligomer ligands is thus prevented, achieving a detection with higher precision and allowing any method that is sensitive to the relevant detection label used to be used as the measuring method, thus also allowing, in this case, non-surface-sensitive detection methods.

### BRIEF DESCRIPTION OF THE DRAWINGS

The invention will be explained in greater detail below by reference to exemplary embodiments in association with the drawings, wherein:

FIG. 1 Shows a schematic diagram of the detection of nucleic acid oligomer hybridization events by means of a displacement assay;

FIG. 2 Shows a cyclovoltammogram of ferrocene carboxylic acid (gold working electrode, platinum counter electrode, Ag/AgCl (sat. KCl) reference electrode, 10 mM ferrocene carboxylic acid);

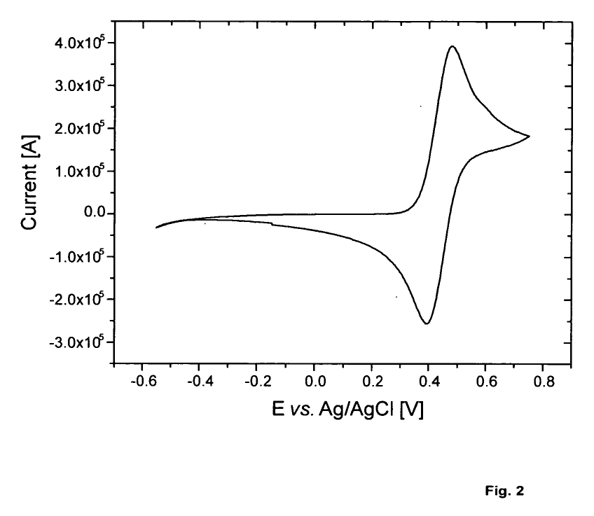

FIG. 3 Shows a chronocoulometric measurement of the sequence-specific hybridization of a 20-mer ligate nucleic acid oligomer with the complementary counter strand (nucleic acid oligomer ligand) by detecting the ferrocene-labeled tetramer signal nucleic acid oligomer ligands displaced by hybridization.

### REFERENCE SIGNS

A: Array having ligate nucleic acid oligomers immobilized via a suitable linker x (two test sites)

B: Array incubated with a solution of signal nucleic acid oligomer ligands

C: Array having signal nucleic acid oligomer ligands associated to ligate nucleic acid oligomers

D: Array having specific complexation of a test site with target

E: Array having specific complexation of a ligate nucleic acid oligomer with target and with signal nucleic acid oligomer ligands associated to a ligate nucleic acid oligomer

101: Ligate nucleic acid oligomer 1

102: Ligate nucleic acid oligomer 2

103: Signal nucleic acid oligomer ligand

104: Detection label, e.g. ferrocene

105: Surface, e.g. gold

106: Nucleic acid oligomer ligand

Step 1: Addition of a signal nucleic acid oligomer ligand

Step 2: Hybridization

Step 3: Dissociation and removal of the signal nucleic acid oligomer ligands and subsequent addition of the nucleic acid oligomer ligand

Step 4: Addition of the nucleic acid oligomer ligand

Step 5: Addition of the signal nucleic acid oligomer ligands

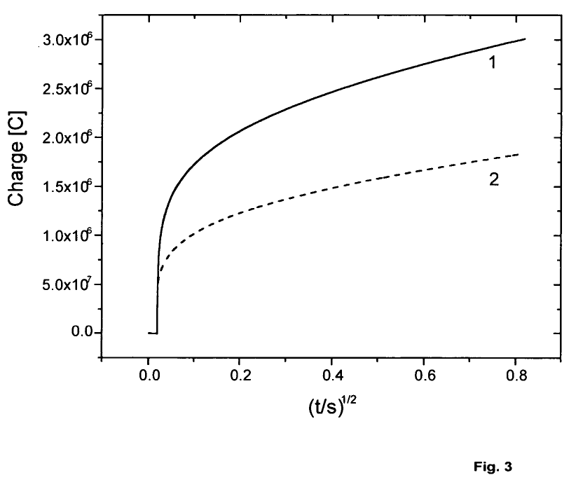

In FIG. 3, the curve labeled “1” displays the chronocoulometric measurement following hybridization of the ferrocene-labeled tetramer signal nucleic acid oligomer ligands, while the curve labeled “2” shows the chronocoulometric measurement following addition of the complementary nucleic acid oligomer ligand and (partial) displacement of the redox-labeled tetramers from the ligate surface.

### Manner of Executing the Invention

Alternative Methods of the Displacement Assay for Detection of Nucleic Acid Oligomer Hybridization Events

To apply the advantages of DNA chip technology to the detection of nucleic acid oligomer hybrids by the displacement assay, various modified ligate nucleic acid oligomers of differing sequence are bound to a support with the above-described immobilization techniques. The arrangement of the ligate nucleic acid oligomers of known sequence at defined positions on the surface, a DNA array, is intended to make the hybridization event of any nucleic acid oligomer ligand or of a (fragmented) ligand DNA detectable in order to e.g. seek and sequence-specifically detect mutations in the nucleic acid oligomer ligand. For this purpose, the surface atoms or molecules of a defined region (a test site) on a surface are linked with DNA/RNA/PNA nucleic acid oligomers of a known but arbitrary sequence as described above. The DNA chip may also be derivatized with a single ligate oligonucleotide. Preferred ligate nucleic acid oligomers are nucleic acid oligomers (e.g. DNA, RNA or PNA fragments) of base length 3 to 80, 3 to 70 or 3 to 50, preferably of length 5 to 50, 8 to 50 or 5 to 30, particularly preferably of length 15 to 35, 10 to 30 or 8 to 25.

The surface thus provided having immobilized ligate oligonucleotides is incubated with a solution of a specific quantity of signal nucleic acid oligomer ligands, e.g. redox-labeled nucleic acid oligomers, that are hybridizable only to a specific sequence section of the ligate oligonucleotide, but not to the entire sequence of the ligate oligonucleotide. This leads to the formation of hybrids comprising ligate nucleic acid oligomer and the signal nucleic acid oligomer ligands in the region of complementary sequences.

Following hybridization between ligate and signal ligand, in a reference measurement, the surface-immobilized portion of the signal nucleic acid oligomer ligands is determined (e.g. by a first chronocoulometric measurement, cf. the section “Surface-sensitive Detection Methods”).

In the next step, the test solution (as concentrated as possible) with ligand oligonucleotide(s) is added to the surface having immobilized ligate oligonucleotides, associated signal nucleic acid oligomer ligands and excess solution (with free, non-surface-adsorbed signal nucleic acid oligomer ligands). This leads to hybridization only if the solution contains ligand nucleic acid oligomer strands that are complementary to the ligate nucleic acid oligomers bound to the surface, or complementary in at least in wide regions (or wider regions than the signal oligonucleotide). The originally associated signal oligonucleotides are, at least partially, displaced.

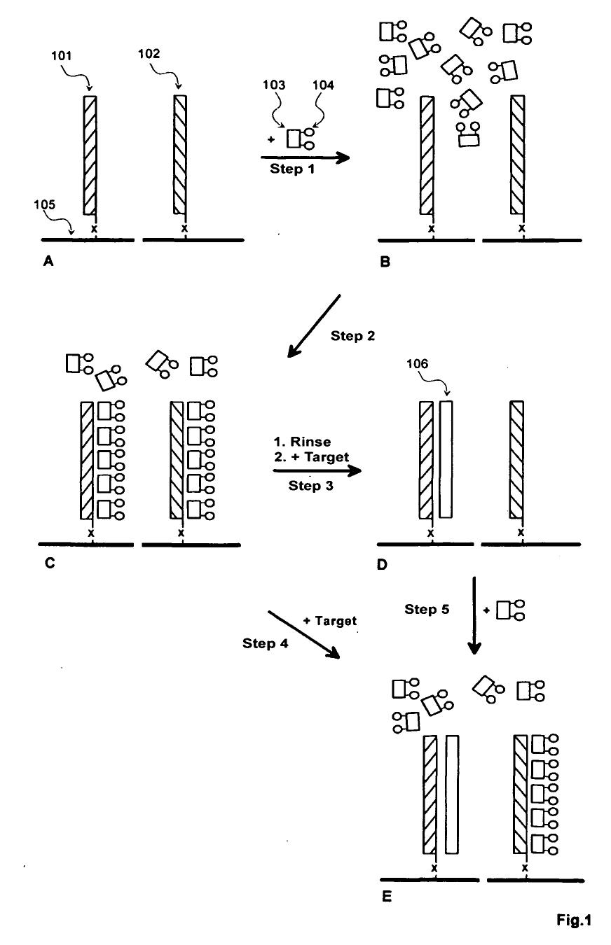

Following hybridization between ligate and ligand, in a second measurement (e.g. a second chronocoulometric measurement, cf. the section “Surface-sensitive Detection Methods”), the portion of remaining surface-immobilized signal nucleic acid oligomer ligands is determined. The difference between reference measurement and second measurement for each test site is proportional to the number of complementary (or complementary in wide regions) ligand oligonucleotides originally present in the test solution for the relevant test site (cf. FIG. 1, procedure with steps 1, 2, 4).

Alternatively, following the reference measurement, the associates comprising ligate nucleic acid oligomer and signal nucleic acid oligomer ligand on the surface may be dissociated, e.g. by increasing the temperature, and all signal nucleic acid oligomer ligands or only the signal nucleic acid oligomer ligands in the excess solution may be removed by washing such that, following the reference measurement, the originally employed surface having immobilized ligate oligonucleotides is available. The removal of the signal nucleic acid oligomer ligands from the associates with the ligate nucleic acid oligomers generally takes place by taking the modified surface out of the solution containing the signal nucleic acid oligomer ligands and subsequently washing the modified surface. In doing so, dehybridizing conditions may be set when washing the modified surface and/or before removing the modified surface from the solution containing the signal nucleic acid oligomer ligands. In the next step, the sample solution with ligand oligonucleotide(s) is added to the surface having immobilized ligate oligonucleotides and the potentially present ligand oligonucleotides may be hybridized to the ligate oligonucleotides under any stringency conditions known to those of ordinary skill in the art. Ideally, by setting the stringency conditions, it can be achieved that exclusively complementary ligand oligonucleotides remain hybridized to the ligate oligonucleotides, whereas “ligand oligonucleotides” that exhibit one or more mismatches dehybridize. Excess test solution with unattached nucleic acid oligomer ligands is removed by washing with suitable buffer solutions. Thereafter—as for carrying out the reference measurement—incubation with the solution containing a specific quantity of signal nucleic acid oligomer ligands again takes place, and in the second measurement, the portion of the signal nucleic acid oligomer ligands still associating to the ligate oligonucleotides is determined (cf. FIG. 1, procedure with steps 1, 2, 3, 5).

In a further alternative, the reference measurement may be omitted if the size of the reference signal is known sufficiently precisely beforehand (e.g. through preceding measurements, etc.). Here, first the hybridization with nucleic acid oligomer ligands and, thereafter, step 5 is carried out (cf. FIG. 1, procedure with step 5). In this case, first the sample solution with ligand oligonucleotides is added to the surface having immobilized ligate oligonucleotides and hybridized, if necessary under strict conditions. Thereafter, a solution that contains a specific quantity of signal nucleic acid oligomer ligands is added. Then, with the aid of a measurement, the portion of signal nucleic acid oligomer ligands that is present associated to the ligate oligonucleotides is determined, and the values obtained are compared with the known reference signal.

### Embodiments

a) Covalent embodiment with attachment of ligate oligonucleotides to (one) individually addressable gold electrode(s), redox-labeled na tetramers as signal nucleic acid oligomer ligands, ligand oligonucleotides and chronocoulometric detection of the displacement of the signal nucleic acid oligomer ligands by the nucleic acid oligomer ligands:

The n-nucleotide (nt)-long ligate nucleic acid (DNA, RNA or PNA, e.g. a 20-nucleotide-long oligo) (FIG. 1A, 101 or 102) is provided near one of its ends (3′- or 5′-end), directly or via a (any) spacer, with a reactive group for covalent anchoring to the surface, e.g. 3′-thiol-modified ligate oligonucleotide in which the terminal thiol modification serves as a reactive group for attachment to gold electrodes. Further covalent anchoring options result from e.g. amino-modified ligate oligonucleotide, which is used for anchoring to glass carbon electrodes that are superficially oxidized onto carboxylic acid, or to platinum electrodes. In addition, a monofunctional linker of suitable chain length with an identical reactive group may be provided. The ligate nucleic acid oligomer modified in this way is,
(i) dissolved in buffer (e.g. 50-500 mM phosphate buffer, pH=7, 1 mM EDTA), brought into contact with the surface and attached there via the reactive group of the ligate nucleic acid oligomer to the—if necessary, appropriately derivatized—surface or
(ii) dissolved in the presence of a monofunctional linker in buffer (e.g. 100 mM phosphate buffer, pH=7, 1 mM EDTA, 0.1-1 M NaCl), brought into contact with the surface and attached there via the reactive group of the ligate nucleic acid oligomer, together with the monofunctional linker, to the—if necessary, appropriately derivatized—surface, care being taken sufficient monofunctional linker of suitable chain length is added (about 0.1- to 10-fold or even 100-fold excess) to provide between the individual ligate oligonucleotides sufficient space for hybridization with the redox-labeled signal nucleic acid oligomer ligands or the ligand oligonucleotide, or
(iii) dissolved in buffer (e.g. 10-350 mM phosphate buffer, pH=7, 1 mM EDTA), brought into contact with the surface and attached there via the reactive group of the ligate nucleic acid oligomer to the—if necessary, appropriately derivatized—surface.

Thereafter, the surface modified in this way is brought into contact with the appropriate monofunctional linker in solution (e.g. alkanethiols or _-hydroxy-alkanethiols in phosphate buffer/EtOH mixtures for thiol-modified ligate oligonucleotides), the monofunctional linker attaching via its reactive group to the—if necessary, appropriately derivatized—surface (cf. the section “The Surface”).

Following appropriate wash steps, the surface modified in this way (FIG. 1A) is brought into contact with redox-labeled signal nucleic acid oligomer ligands (FIG. 1B, 104) comprising fewer than n complementary nucleotides (FIG. 1B, 103). As redox-labeled signal nucleic acid oligomer ligands, e.g. singly or multiply ferrocene-carboxylic acid-modified nucleic acid tetramers (cf. ex. 3) whose sequence is complementary to tetramer partial sequences of the ligate oligonucleotides may be used, or SSB (single-stranded DNA-binding protein) modified with redox label (e.g. ferrocene derivatives) may be employed. Here, care is taken that significantly more labeled signal nucleic acid oligomer ligands (at least 1.1-fold molar excess) are added than can be bound to the surface via the ligate nucleic acid oligomers.

The detection label on the signal nucleic acid oligomer ligand is detected by a suitable method, e.g. chronocoulometry in the case of the ferrocene-redox-labeled signal oligonucleotides. Thereafter, the dissolved ligand is added and the measurement for detecting the detection label is repeated with the suitable method (e.g. renewed chronocoulometric measurement in the case of the ferrocene-redox-labeled signal oligonucleotides).

Alternatively, the modified surface is removed from the solution containing signal nucleic acid oligomer ligands, if necessary washed as described above and subsequently brought into contact with the solution containing the nucleic acid oligomer ligands. The hybridization may be carried out under suitable conditions known to those of ordinary skill in the art (any, freely selectable stringency conditions for the parameters potential/temperature/salt/chaotropic salts, etc., for hybridization). Thereafter, the modified surface is brought into contact with signal nucleic acid oligomer ligands (in the same concentration as for the preceding measurement) and a measurement for detecting the detection label is carried out with a suitable method.

The difference in the measurement signal (decrease or increase, depending on the measuring method) is proportional to the number of hybridization events between ligate nucleic acid oligomer on the surface and matching nucleic acid oligomer ligand in the test solution (cf. ex. 6). In chronocoulometric measurement, a decrease is detected in the nearly instantaneous signal portion of the surface excess of redox labels, cf. “Surface-sensitive Detection Methods.”

As a variant of the described method, after the first detection (reference measurement) has taken place, the complexes comprising surface-bound ligate nucleic acid oligomer and labeled signal nucleic acid oligomer ligands (FIG. 1C) are dissolved (e.g. by temperature increase) and the free, labeled signal nucleic acid oligomer ligands are removed from the excess by washing (FIG. 1, \textcircled{3}). Following addition of the nucleic acid oligomer ligand, hybridization (FIG. 1D) and addition of the labeled signal nucleic acid oligomer ligands (FIG. 1, \textcircled{5}), a second chronocoulometric measurement (FIG. 1E) is carried out.

The method may be applied for one ligand type, i.e. a specific ligand oligonucleotide type having a known sequence, on one electrode; or for multiple ligand types, i.e. differing ligand oligonucleotide types, on individually addressable electrodes of an electrode array that can be targeted and read out, e.g. via CMOS technology, in more complex arrays.

b) Embodiment with indirect attachment of ligate oligonucleotides to glass fibers, with fluorophore-labeled na dodecamers as signal nucleic acid oligomer ligands, oligonucleotide ligands and fluorescence detection of the displacement of the signal nucleic acid oligomer ligands by the nucleic acid oligomer ligands:

The n-nucleotide-long ligate nucleic acid (DNA, RNA or PNA) (FIG. 1A, 101 or 102, e.g. a 20-nucleotide-long oligo), is provided near one of its ends (3′- or 5′-end), directly or via a (any) spacer, with a reactive group for covalent anchoring to the surface, e.g. a carboxy-modified ligate oligonucleotide for attachment to amino-modified silanized glass (e.g. to (3-aminopropyl)-triethoxysilane-modified glass). Further covalent anchoring options result from e.g. amino-modified ligate oligonucleotide, which is used for anchoring to dextran polymers that are derivatized with carboxylic acid and immobilized on glass, the thickness of the layer comprising dextran polymer with attached ligate oligonucleotides in this embodiment being able to be varied via the dextran polymer composition, dextran anchor groups on the glass surface, anchor groups of the dextran for immobilization on the glass, incubation duration on the glass, etc., using methods known to those of ordinary skill in the art. In a preferred embodiment, the thickness of the dextran/ligate oligonucleotide layer is chosen such that it approximately corresponds to the penetration depth of the evanescent field of the light for the excitation of the fluorophores (approx. 50 nm to approx. 500 nm, depending on whether a metallization layer is located on the glass to increase the penetration depth of the evanescent field). The ligate nucleic acid modified in this way is dissolved in buffer (e.g. 50-500 mM phosphate buffer, pH=7, 1 mM EDTA) in the presence of EDC and sNHS (each approximately 40-fold molar excess in reference to-the ligate oligonucleotide), brought into contact with the modified glass surface and attached there to the surface via the reactive group of the ligate nucleic acid oligomer (if necessary, nonfunctional surface binding sites are treated beforehand with a suitable blocking reagent).

Following suitable wash steps, the surface modified in this way (FIG. 1A) is brought into contact with fluorophore-labeled signal nucleic acid oligomer ligands (FIG. 1B, 104) comprising fewer than n complementary nucleotides (FIG. 1B, 103). As fluorophore-labeled signal nucleic acid oligomer ligands, e.g. fluorescein-modified nucleic acid dodecamers may be used whose sequences are complementary to dodecamer partial sequences of the ligate oligonucleotides, or SSB (single-stranded DNA-binding protein) modified with one or more fluoresceins (e.g. FITC derivatives) may be employed. Here, care is taken that significantly more labeled signal nucleic acid oligomer ligands (at least 1.1-fold molar excess) are added than can be bound to the surface via the ligate nucleic acid oligomers.

The detection label on the signal ligand oligonucleotide is detected by a suitable method, e.g. total internal reflection fluorescence (TIRF) in the case of the fluorophore-labeled signal nucleic acid oligomer ligands. Thereafter, the signal nucleic acid oligomer ligand in the excess solution is removed by washing, the test solution is added and potential hybridization events are facilitated under suitable conditions known to those of ordinary skill in the art (any, freely selectable stringency conditions for the parameters potential/temperature/salt/chaotropic salts, etc., for hybridization). After that, the quantity of signal ligand originally employed for the reference measurement is again added and the measurement for detecting the detection label is repeated with the suitable method (e.g. renewed TIRF measurement in the case of the fluorescein-labeled signal nucleic acid oligomer ligands). The difference in the measurement signal (decrease or increase, depending on the measuring method) is proportional to the number of hybridization events between ligate nucleic acid oligomer on the surface and matching nucleic acid oligomer ligand in the test solution (cf. ex. 6). For detection by determining the TIRF, a decrease in the fluorescence signal is to be expected.

As a variant of the described method, after the first detection (reference measurement) has taken place, the complexes comprising surface-bound ligate nucleic acid oligomer and labeled signal nucleic acid oligomer ligands (FIG. 1C) are dissolved and the free, labeled oligonucleotides are removed from the excess by washing (FIG. 1, \textcircled{3}). Following addition of the nucleic acid oligomer ligands, hybridization (FIG. 1D) and addition of the labeled signal nucleic acid oligomer ligands (FIG. 1, \textcircled{5}), a second measurement (FIG. 1E) is then carried out.

The method may be applied for one ligand type, i.e. a specific ligand oligonucleotide type having a known sequence, on a glass fiber; or for multiple ligand types, i.e. differing ligand oligonucleotide types, on individually addressable glass fibers of a glass fiber bundle.

c) SNP embodiment with attachment of ligate oligonucleotides comprising 20 nucleotides to (one) individually addressable gold electrode(s), redox-labeled SNP-ID ligands comprising 19 nucleotides whose sequence is complementary to the sequence of the ligate nucleic acid oligomers, ligand oligonucleotides and cyclovoltammetric detection of the signal nucleic acid oligomer ligands:

The 20-nucleotide (nt)-long ligate nucleic acid (DNA, RNA or PNA, FIG. 1A, 101 or 102) is provided near one of its ends (3′- or 5′-end), directly or via a (any) spacer, with a reactive group for covalent anchoring to the surface, e.g. 3′-thiol-modified ligate oligonucleotide in which the terminal thiol modification serves as a reactive group for attachment to gold electrodes. The ligate nucleic acid oligomer modified in this way is,
(i) dissolved in buffer (e.g. 50-500 mM phosphate buffer, pH=7, 1 mM EDTA), brought into contact with the surface and attached there via the reactive group of the ligate nucleic acid oligomer to the—if necessary, appropriately derivatized—surface or
(ii) dissolved in the presence of a monofunctional linker in buffer (e.g. 100 mM phosphate buffer, pH=7, 1 mM EDTA, 0.1-1 M NaCl), brought into contact with the surface and attached there via the reactive group of the ligate nucleic acid oligomer, together with the monofunctional linker, to the—if necessary, appropriately derivatized—surface, care being taken that sufficient monofunctional linker of suitable chain length is added (about 0.1- to 10-fold or even 100-fold excess) to provide between the individual ligate oligonucleotides sufficient space for hybridization with the redox-labeled signal nucleic acid oligomer ligands or the ligand oligonucleotide, or
(iii) dissolved in buffer (e.g. 10-350 mM phosphate buffer, pH=7, 1 mM EDTA), brought into contact with the surface and attached there via the reactive group of the ligate nucleic acid oligomer to the—if necessary, appropriately derivatized—surface. Thereafter, the surface modified in this way is brought into contact with the appropriate monofunctional linker in solution (e.g. alkanethiols or _-hydroxy-alkanethiols in phosphate buffer/EtOH mixtures for thiol-modified ligate oligonucleotides), the monofunctional linker attaching via its reactive group to the—if necessary, appropriately derivatized—surface (cf. the section “The Surface”).

Following appropriate wash steps, the surface modified in this way (FIG. 1A) is brought into contact with the nucleic acid ligands. After a 30-minute reaction time, the modified surface is rinsed with buffer (e.g. 350 mM phosphate buffer, pH=7, 1 mM EDTA) to remove from the surface and the excess solution nucleic acid oligomers that are not associated to ligate nucleic acid oligomers. Thereafter, redox-labeled SNP-ID ligands comprising 19 nucleotides whose sequence is complementary to the sequence of the ligate nucleic acid oligomers, are brought into contact with the surface. Covalently attached ferrocene carboxylic acid serves as the redox label. Here, the quantity of SNP-ID ligands is set such that the molar ratio of SNP-ID ligands and ligate nucleic acid oligomers is 2-5 and the concentration of the SNP-ID ligand is as high as possible. After a 30-minute reaction time, the modified surface is rinsed with buffer (e.g. 350 mM phosphate buffer, pH=7, 1 mM EDTA) to remove from the surface and the excess solution SNP-ID ligands that are not associated to ligate nucleic acid oligomers.

The detection label on the SNP-ID ligand is detected by a suitable method, e.g. cyclovoltammetric determination of the ferrocene-redox-labeled signal oligonucleotides. The value obtained is compared with a previously established reference value, for which the SNP-ID ligand was associated under identical hybridization conditions (quantity, concentration, hybridization duration) to an identical modified surface, but in the absence of nucleic acid oligomer ligands.

If the measurement signal differs significantly from the reference value (more than 50% decrease), the appropriate nucleic acid oligomer ligand that is complementary to the ligate nucleic acid oligomer was present, and namely as a perfect match for the partial region, comprising 20 nucleotides, that the ligate can cover.

### EXAMPLE 1

### Constituting the N-hydroxysuccinimide active ester of the redox (or fluorophore) label

1 mmol of the relevant carboxylic acid derivative of a fluorophore (e.g. fluorescein) or of a redox-active substance (e.g. ferrocene) and 1.1 mmol N-hydroxysuccinimide are dissolved in 15 ml anhydrous dioxane. 1.1 mmol carbodiimide (dissolved in 3 ml anhydrous dioxane) are cooled with ice and added dropwise to the carboxylic acid derivative. The reaction mixture is stirred for 16 h at RT, the resultant precipitate filtered off and the solvent drawn off. The residue is purified by silica gel chromatography (Merck silica gel 60, solvent system: dichloromethane/ethyl acetate/heptane mixtures).

### EXAMPLE 2

### Constituting the amino-modified oligonucleotides for coupling the active ester label of ex. 1, or thiol-modified oligonucleotides for anchoring on gold as ligate nucleic acid oligomers

The synthesis of the oligonucleotides takes place in an automatic oligonucleotide synthesizer (Expedite 8909; ABI 384 DNA/RNA Synthesizer) according to the synthesis protocol recommended by the manufacturer for a 1.0 μmol synthesis.

By default, the synthesis of the (signal) nucleic acid oligomer ligands takes place on A-CPG as the support material. Modifications at the 5′-position of the oligonucleotides take place with a coupling step prolonged to 5 minutes. The amino modifier C2 dT (Glen Research 10-1037) is incorporated into the sequences with the relevant standard protocol.

The constitution of 3′-thiol-modified ligate oligonucleotides (or HO—(CH₂)₂—SS—(CH₂)₂OPO₃ oligonucleotides) takes place on 1-O-dimethoxytrityl-propyl-disulfide-CPG support (Glen Research 20-2933) analogously to standard protocols, the oxidation steps being carried out with a 0.02 M iodine solution to avoid oxidative cleavage of the disulfide bridge.

During synthesis, the coupling efficiencies are determined online photometrically or conductometrically via the DMT cation concentration.

The oligonucleotides are deprotected with concentrated ammonia (30%) at 37° C. over a period of 16 h. The purification of the oligonucleotides takes place by means of RP-HPL chromatography according to standard protocols (solvent system: 0.1 M triethylammonium acetate buffer, acetonitrile), the characterization by MALDI-TOF MS.

### EXAMPLE 3

### Converting the amino-modified oligonucleotides (ex. 2) with the N-hydroxy active esters (ex. 1)

The amino-modified oligonucleotides are dissolved in 0.1 M borate buffer (pH 8.5) and converted with the N-hydroxysuccinimide active esters dissolved in DMSO according to the protocol from Molecular Probes (Labeling Amine-Modified Oligonucleotides). The purification of the oligonucleotides takes place by means of RP-HPL chromatography according to standard protocols (solvent system: 0.1 M triethylammonium acetate buffer, acetonitrile), the characterization by MALDI-TOF MS.

### EXAMPLE 4

### Producing the oligonucleotide electrode Au—S(CH₂)₂-ss-oligo

Au—S(CH₂)₂-ss-oligo is produced in 2 steps, namely constituting the conductive surface and derivatizing the surface with the ligate oligonucleotide in the presence of a suitable monofunctional linker (incubation step).

An approx. 100 nm thin gold film on mica (muscovite lamina) forms the support material for the covalent attachment of the double-strand oligonucleotides. For this purpose, freshly cleaved mica is purified with an argon-ion plasma in an electrical discharge chamber and gold (99.99%) is applied by electrical discharge in a layer thickness of approx. 100 nm. Thereafter, the gold film is freed of surface impurities (oxidation of organic accumulations) with 30% H₂O₂/70% H₂SO₄ and immersed in ethanol for approx. 20 minutes to dispel any oxygen adsorbed on the surface. After rinsing the surface with bidistilled water, a previously prepared 1×10⁻⁴ molar solution of the (modified) oligonucleotide is applied onto the horizontally mounted surface, such that the entire gold surface is wetted (incubation step, see also below).

For incubation, a modified 20-bp single-strand oligonucleotide having the sequence 5′-TAG CGG ATA ACA CAG TCA CC-3′ is used, which is esterified with (HO—(CH₂)₂—S)₂ at the phosphate group of the 3′-end to form P—O—(CH₂)₂—S—S—(CH₂)₂—OH (cf. ex. 2). An approx. 10⁻⁵ to 10⁻¹ molar propanethiol solution (or another thiol or disulfide of suitable chain length) is added to a 5×10⁻⁵ molar solution of this oligonucleotide in HEPES buffer (0.1 molar in water, pH 7.5 with 0.7 molar addition of TEA TFB), the gold surface of a test site completely wetted and incubated for 2-24 hours. During this reaction time, the disulfide spacer P—O—(CH₂)₂—S—S—(CH₂)₂—OH of the oligonucleotide is homolytically cleaved. In this process, the spacer forms a covalent Au—S bond with Au atoms of the surface, thus causing a 1:1 coadsorption of the ss-oligonucleotide and the cleaved 2-hydroxy-mercaptoethanol. The free propanethiol that is also present in the incubation solution is likewise coadsorbed by forming an Au—S bond (incubation step). Instead of the single-strand oligonucleotide, this single-strand may also be hybridized with its unmodified complementary strand.

### EXAMPLE 5

### Alternative production of the oligonucleotide electrode Au—S(CH₂)₂-ss-oligo

Au—S(CH₂)₂-ss-oligo is alternatively produced in 3 steps, namely constituting the conductive surface, derivatizing the surface with the ligate oligonucleotide (incubation step) and postcoating the electrode modified in this way with a suitable monofunctional linker (postcoating step).

An approx. 100 nm thin gold film on mica (muscovite lamina) forms the support material for the covalent attachment of the ligate oligonucleotides, cf. ex. 4.

For incubation, a modified 20-bp single-strand oligonucleotide having the sequence 5′-TAG CGG ATA ACA CAG TCA CC-3′ is used, which is esterified with (HO—(CH₂)₂—S)₂ at the phosphate group of the 3′-end to form P—O—(CH₂)₂—S—S—(CH₂)₂—OH. The gold surface of a test site is wetted with an approx. 5×10⁻⁵ molar solution of this oligonucleotide in HEPES buffer (0.1 molar in water, pH 7.5) and incubated for 2-24 hours. During this reaction time, the disulfide spacer P—O—(CH₂)₂—S—S—(CH₂)₂—OH of the oligonucleotide is homolytically cleaved. In this process, the spacer forms a covalent Au—S bond with Au atoms of the surface, thus causing a coadsorption of the ss-oligonucleotide and the cleaved 2-hydroxy-mercaptoethanol (incubation step).

Thereafter, the gold electrode modified in this way is completely wetted with an approx. 10⁻⁵ to 10⁻¹ molar propanethiol solution (in water or buffer, pH 7-7.5) or with another thiol or disulfide (of suitable chain length) and incubated for 2-24 hours. After the incubation step, the free propanethiol coats remaining free gold surface by forming an Au—S bond.

### EXAMPLE 6

### Chronocoulometric measurement in the Au-ss-oligo/ferrocene-modified nucleic acid tetramer system in the absence and presence of nucleic acid oligomer ligands (complementary to ss-oligo in Au-ss-oligo)

A probe electrode is produced according to ex. 5. For this purpose, the above-described HO—(CH₂)₂—SS—(CH₂)₂-modified oligonucleotide (sequence TAG CGG ATA ACA CAG TCA CC) is immobilized on gold (50 μmol oligonucleotide in phosphate buffer (500 mM K₂HPO₄/KH₂PO₄ pH 7), postcoating with 1 mM propanethiol in water).

Following addition of complementary double ferrocene-labeled nucleic acid tetramers (10 μM), a potential jump experiment is carried out. The values obtained by chronocoulometric measurement are shown in FIG. 3, curve 1. Following addition of the complementary target (5 μM), the potential jump experiment is repeated. The values obtained from the chronocoulometric measurement repeated subsequently are shown in FIG. 3, curve 2.

The diameter of the gold electrode used measures 6 mm, i.e. a surface area of 0.28 cm²is available for the immobilization of the ligate nucleic acid oligomers. The integrals of curves 1 and 2 (FIG. 3) yield a difference of 70×10⁻⁸ C (0.7 μC). This value corresponds to 2.5 μC/cm² or 1.6×10¹³ electrons/cm². Assuming maximum coverage with ligate oligonucleotides, in other words coverage with 7×10¹² ligate oligonucleotides per cm², an average of at least approx. 2.2 electrons per ligate oligonucleotide were thus converted, i.e. 2.2 ferrocene labels were displaced by hybridization of the ligate nucleic acid oligomer with the nucleic acid oligomer ligand. Thus, on average, 1.1 tetramers were displaced from the ligate oligonucleotide.

1. A method for detection of nucleic acid oligomer hybridization events, comprising the steps
a) providing a modified surface, the modification consisting in the attachment of at least one type of ligate nucleic acid oligomer,
b) providing signal nucleic acid oligomer ligands,
c) providing a sample having nucleic acid oligomer ligands,
d) bringing a defined quantity of the signal nucleic acid oligomer ligands into contact with the modified surface and bringing the sample and the nucleic acid oligomer ligands contained therein into contact with the modified surface,
e) detecting the signal nucleic acid oligomer ligands,
f) comparing the values obtained in step e) with reference values.

2. The method according to claim 1, wherein in step d), bringing a defined quantity of the signal nucleic acid oligomer ligands into contact with the modified surface and bringing the sample into contact with the modified surface take place simultaneously.

3. The method according to claim 1, wherein in step d), bringing a defined quantity of the signal nucleic acid oligomer ligands into contact with the modified surface and bringing the sample into contact with the modified surface take place separately.

4. The method according to claim 3, wherein as step d), first the step
d₁) bringing a defined quantity of the signal nucleic acid oligomer ligands into contact with the modified surface
and thereafter the step
d₂) bringing the sample into contact with the modified surface
is carried out.

5. The method according to claim 4, wherein after step d₁) and before step d₂) the step
d₃) detecting the signal nucleic acid oligomer ligands
is carried out and in step f) the values obtained in step e) are compared with the reference values obtained in step d₃).

6. The method according to claim 5, wherein after step d₃) and before step d₂) the step
d₄) washing the modified surface
is carried out, and after step d₂) and before step e) the step
d₅) bringing the signal nucleic acid oligomer ligands into contact with the modified surface, the identical defined quantity of signal nucleic acid oligomer ligands being used as in step d₁)
is carried out.

7. The method according to claim 6, wherein after step d₃) and before or during step d₄) the step
d₆) setting conditions or taking actions that lead to at least predominant dissociation of ligate nucleic acid oligomers and signal nucleic acid oligomer ligands
is carried out.

8. The method according to claim 7, wherein in step d₆), the temperature is raised above the melting temperature of the double-stranded oligonucleotides consisting of ligate nucleic acid oligomer and signal nucleic acid oligomer ligand.

9. The method according to claim 7, wherein in step d₆), chaotropic salts are added.

10. The method according to claim 7, wherein in step d₆), a potential that lies above the electrostringent potential is applied.

11. The method according to claim 2, wherein after step e) the steps
e₁) setting conditions or taking actions that lead to at least predominant dissociation of ligate nucleic acid oligomers and nucleic acid oligomer ligands, and to at least predominant dissociation of ligate nucleic acid oligomers and signal nucleic acid oligomer ligands, washing the modified surface,
e₂) bringing the signal nucleic acid oligomer ligands into contact with the modified surface, the identical defined quantity of signal nucleic acid oligomer ligands being used as in step d),
e₃) detecting the signal nucleic acid oligomer ligands
are carried out and in step f), the values obtained in step e) are compared with the reference values obtained in step e₃).

12. The method according to claim 11, wherein in step e₁), the temperature is raised above the melting temperature of the double-stranded oligonucleotides consisting of ligate nucleic acid oligomers and nucleic acid oligomer ligands, and above the melting temperature of the double-stranded oligonucleotides consisting of ligate nucleic acid oligomers and signal nucleic acid oligomer ligands.

13. The method according to claim 11, wherein in step e₁), chaotropic salts are added.

14. The method according to claim 11, wherein in step e₁), a potential that lies above the electrostringent potential is applied.

15. The method according to one of claim 11, wherein before step e₃) the step
e₄) washing the modified surface
is carried out.

16. The method according to claim 3, wherein as step d), first the step
d₂) bringing the sample into contact with the modified surface
and thereafter the step
d₁) bringing a defined quantity of the signal nucleic acid oligomer ligands into contact with the modified surface
is carried out.

17. The method according to claim 16, wherein after step e) the steps
e₁) setting conditions or taking actions that lead to at least predominant dissociation of ligate nucleic acid oligomers and nucleic acid oligomer ligands, and to at least predominant dissociation of ligate nucleic acid oligomers and signal nucleic acid oligomer ligands, washing the modified surface,
e₂) bringing the signal nucleic acid oligomer ligands into contact with the modified surface, the identical defined quantity of signal nucleic acid oligomer ligands being used as in step d₁),
e₃) detecting the signal nucleic acid oligomer ligands
are carried out and in step f), the values obtained in step e) are compared with the reference values obtained in step e₃).

18. The method according to claim 17, wherein in step e₁), the temperature is raised above the melting temperature of the double-stranded oligonucleotides consisting of ligate nucleic acid oligomers and nucleic acid oligomer ligands, and above the melting temperature of the double-stranded oligonucleotides consisting of ligate nucleic acid oligomers and signal nucleic acid oligomer ligands.

19. The method according to claim 17, wherein in step e₁), chaotropic salts are added.

20. The method according to claim 17, wherein in step e₁), a potential that lies above the electrostringent potential is applied.

21. The method according to claim 2, wherein as step a) the step
a) providing a modified surface, the modification consisting in the attachment of at least two types of ligate nucleic acid oligomers, and the differing types of ligate nucleic acid oligomers being bound to the surface in spatially substantially separate regions
is carried out, after step c) and before step d) the step
c₁) adding one type of nucleic acid oligomer ligand to the sample, the nucleic acid oligomer ligand being a binding partner having a high association constant of a type of ligate nucleic acid oligomer that is bound to the surface in a specific region T₁₀₀, the nucleic acid oligomer ligand being added in a quantity that is greater than the quantity of nucleic acid oligomer ligands needed to completely associate the ligate nucleic acid oligomers of the T₁₀₀ test sites
is carried out and in step f), the values obtained in step e) are compared with the value obtained for the T₁₀₀ region.

22. The method according to claim 21, wherein as step a) the step
a) providing a modified surface, the modification consisting in the attachment of at least three types of ligate nucleic acid oligomers, and the differing types of ligate nucleic acid oligomers are bound to the surface in spatially substantially separate regions, at least one type of ligate nucleic acid oligomer being attached to the surface in a specific region T₀, and no binding partner having a high association constant to this type of ligate nucleic acid oligomer is contained in the sample
is carried out and in step f), the values obtained in step e) are compared with the value obtained for the T₁₀₀ region, and with the value obtained for the T₀ region.

23. The method according to claim 21, wherein before step d) and after step c₁) the step
c₂) adding at least one additional type of nucleic acid oligomer ligand to the sample, the nucleic acid oligomer ligand in the sample provided in step c) not being contained, and the nucleic acid oligomer ligand exhibiting an association constant>0 to a type of ligate nucleic acid oligomer that is bound to the surface in a specific region Tₙ, the nucleic acid oligomer ligand being added in a quantity such that, after step d), n % of the ligate nucleic acid oligomers in the Tₙ region are present in associated form
is carried out and in step f), the values obtained in step e) are compared with the value obtained for the T₁₀₀ region, with the value obtained for the T₀ region and with the values obtained for the Tₙ regions.

24. The method according to claim 21, wherein the signal nucleic acid oligomer ligands are added in a quantity that is greater than the quantity of signal nucleic acid oligomer ligands needed to completely associate the ligate nucleic acid oligomers of the T₁₀₀ test sites.

25. The method according to claim 1, wherein the signal nucleic acid oligomer ligands are modified with a detection label.

26. The method according to claim 1, wherein the signal nucleic acid oligomer ligands are modified with multiple detection labels.

27. The method according to claim 25, wherein a fluorophore is used as the detection label, especially a fluorescent dye, especially Texas Red, a rhodamine dye or fluorescein.

28. The method according to claim 25, wherein a redox-active substance is used as the detection label.

29. The method according to claim 1, wherein the modified surface is a conductive surface.

30. The method according to claim 1, wherein the detection of the signal nucleic acid oligomer ligands takes place through a surface-sensitive detection method.

31. The method according to claim 30, wherein the detection of the signal nucleic acid oligomer ligands takes place through a spectroscopic, an electrochemical or an electrochemiluminescent method.

32. The method according to claim 31, wherein the spectroscopic detection takes place through detecting the fluorescence, especially the total internal reflection fluorescence (TIRF), of the signal nucleic acid oligomer ligands.

33. The method according to claim 31, wherein the electrochemical detection takes place through amperometry, chronocoulometry, impedance measurement or scanning electrochemical microscopy (SECM).

34. The method according to claim 1, wherein the nucleic acid oligomers used as ligate nucleic acid oligomers comprise 3 to 80 nucleic acids, especially 5 to 50 nucleic acids, particularly preferably 15 to 35 or 8 to 25 nucleic acids.

35. The method according to claim 1, wherein signal nucleic acid oligomer ligands are used that consist of n or more nucleotides and exhibit only such regions made up of n nucleotides whose sequence is complementary to the sequence of the ligate nucleic acid oligomer in fewer than n nucleotides, especially in n-1, n-2, n-3, n-4 or n-5 nucleotides, the ligate nucleic acid oligomer comprising n nucleotides, and n being an integer from 6 to 80, especially from 6 to 50, particularly preferably from 15 to 35 or from 8 to 25.

36. The method according to claim 1, wherein every type of signal nucleic acid oligomer ligand exhibits at least one sequence section that consists of a maximum of n-1, n-2, n-3, n-4 or n-5 nucleotides and that is complementary to one sequence section of the ligate nucleic acid oligomer, the ligate nucleic acid oligomer comprising n nucleotides, and n being an integer from 6 to 80, especially from 6 to 50, particularly preferably from 15 to 35 or from 8 to 25.

37. The method according to claim 1, wherein every type of signal nucleic acid oligomer ligand exhibits multiple partial-sequence sections, the multiple partial-sequence sections all together consisting of a total of n-1, n-2, n-3, n-4 or n-5 nucleotides, and the multiple partial-sequence sections being complementary to one sequence section of the ligate nucleic acid oligomer, the ligate nucleic acid oligomer comprising n nucleotides, and n being an integer from 6 to 80, especially from 6 to 50, particularly preferably from 15 to 35 or from 8 to 25.

38. The method according to claim 1, wherein each time, one type of ligate nucleic acid oligomer consists of a number of n nucleotides, and the signal nucleic acid oligomer ligands that are complementary to this type of ligate nucleic acid oligomer consist of a number of n-1, n-2, n-3, n-4 or n-5 nucleotides, n being an integer from 6 to 80, especially from 6 to 50, particularly preferably from 15 to 35 or from 8 to 25.

39. The method according to claim 35, wherein, each time, one type of nucleic acid oligomer ligand exhibits a nucleotide that is not complementary to the corresponding nucleotide of the complementary type of ligate nucleic acid oligomer, and wherein all other nucleotides of said type of nucleic acid oligomer ligand are complementary to the corresponding nucleotides of the complementary type of ligate nucleic acid oligomer.

40. The method according to claim 35, wherein before step d₃) the step
d₇) washing the modified surface
is carried out.

41. The method according to claim 35, wherein before step e₃) the step
e₄) washing the modified surface
is carried out.

42. The method according to claim 35, wherein before step e) the step
d₈) washing the modified surface
is carried out.

43. The method according to claim 21, wherein the differing types of ligate nucleic acid oligomers each exhibit a different number of nucleic acids.

44. The method according to claim 1, wherein differing types of signal nucleic acid oligomer ligands are used.

45. The method according to claim 44, wherein the differing types of signal nucleic acid oligomer ligands each exhibit a different number of nucleotides.

46. The method according to claim 1, wherein the molar ratio of signal nucleic acid oligomer ligands to ligate nucleic acid oligomers is between 0.01 and 1000, preferably between 0.1 and 100, particularly preferably between 1 and 10.

47. The method according to claim 1, wherein signal PNA oligomer ligands are used as signal nucleic acid oligomer ligands.

48. A kit for carrying out a method according to claim 1, comprising a modified surface, the modification consisting in the attachment of at least one type of ligate nucleic acid oligomer; and an effective quantity of signal nucleic acid oligomer ligands as defined in claims 1 to 47.

49. The kit according to claim 48, wherein the kit additionally comprises reference values for comparison with the values obtained from the detection of the signal nucleic acid oligomer ligands in a method according to claims 1 to 47.

50. The kit according to claim 48, wherein the modified surface comprises at least one T₀ region as defined in claim 22, and at least one T₁₀₀ region as defined in claim 21.

51. The kit according to claim 50, wherein the modified surface additionally comprises at least one Tₙ region as defined in claim 23.
